(19) United States
(12) Patent Application Publication
(10) Pub. No.: US 2006/0293288 A1
(43) Pub. Date: Dec. 28, 2006

(54) Use of resolvins to treat gastrointestinal diseases

(76) Inventors: Charles N. Serhan, Needham, MA (US); Makoto Arita, Brookline, MA (US); Masaru Yoshida, Newton, MA (US); Richard Blumberg, Chestnut Hill, MA (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/326,134

(22) Filed: Jan. 5, 2006

Related U.S. Application Data
(60) Provisional application No. 60/642,056, filed on Jan. 7, 2005.

(51) Int. Cl. A61K 31/655; A61K 31/5375; A61K 31/551; A61K 31/496; A61K 31/445; A61K 31/277; A61K 31/275; A61K 31/20; A61K 31/18; A61K 31/16; A61K 31/10

(52) U.S. Cl. 514/149; 514/218; 514/231.2; 514/317; 514/252.12; 514/560; 514/150; 514/519; 514/601; 514/625; 514/740; 514/514; 514/708; 514/640

(57) ABSTRACT

The present invention is generally drawn to novel isolated therapeutic agents, termed resolvins, generated from the interaction between a dietary omega-3 polyunsaturated fatty acid (PUFA) such as eicosapentaenoic acid (EPA) or docosahexaenoic acid (DHA), oxygenases and the analgesic aspirin (ASA). Surprisingly, careful isolation of compounds generated from the combination of components in an appropriate environment provide di- and tri-hydroxy containing derivatives of EPA and DHA containing compounds having unique structural and physiological properties. The present invention therefore provides for many new useful therapeutic di- and tri-hydroxy derivatives of EPA or DHA (resolvins of the E series and D series) that diminish, prevent, or eliminate gastrointestinal conditions, for example, such as colitis. The present invention also provides methods of use, methods of preparation, and packaged pharmaceuticals for use as medicaments for the compounds disclosed throughout the specification.

### CROSS REFERENCE TO RELATED APPLICATIONS

This application claims benefit under 35 U.S.C. § 119(e) to U.S. Application Ser. No. 60/642,056, filed Jan. 7, 2005, the contents of which are incorporated herein in their entirety.

### STATEMENT REGARDING FEDERALLY SPONSORED RESEARCH

The work leading to this invention was supported in part by National Institutes of Health (NIH) grants GM38765 and P01-DE13499. The U.S. Government therefore may have certain rights in the invention.

### FIELD OF THE INVENTION

The present invention relates to previously unknown therapeutic agents derived from novel signaling and biochemical pathways that use eicosapentaenoic acid (EPA) and/or docosahexaenoic acid (DHA), both of which are polyunsaturated fatty acids (PUFAs) as precursors to the production of bioactive novel endogenous products that control physiologic events in inflammation and resolution in vascular endothelial reactions and neural systems (brain). More specifically, the present invention relates to di- and trihydroxy potent bioactive products termed “Resolvins” and “Protectins” which are derived from polyunsaturated fatty acids. In addition, therapeutic stable analogs of resolvins of the E and D series and protectins that could enhance their biologic properties are described that can be used to expedite resolution by inhibiting the pro-inflammatory amplification of leukocyte entry.

### BACKGROUND OF THE INVENTION

In many chronic disorders, unresolved inflammation is a major mechanism of disease pathogenesis (1). Inflammation is a protective host response to foreign antigenic challenge or tissue injury that could lead to, if unopposed, loss of tissue structure as well as function. During the development of inflammation, the concerted actions of molecular signaling determine whether inflammatory cells undergo migration, activation, proliferation, differentiation or clearance. Many inflammatory processes are self-limited and self-resolving systems, suggesting the existence of endogenous anti-inflammatory and/or pro-resolution mediators during the course of inflammation (for recent reviews, see 6, 15-17).

Omega-3 polyunsaturated fatty acids (PUFA) such as eicosapentaenoic acid (EPA) and docosahexaenoic acid (DHA) which are enriched in fish oils are held to be beneficial in a wide range of human inflammatory disorders including cardiovascular diseases, rheumatoid arthritis, Alzheimer's disease, lung fibrosis and inflammatory bowel disease (2-5). These essential fatty acids are widely believed to act via several possible mechanisms, such as preventing conversion of arachidonate to proinflammatory eicosanoids, or serving as an alternative substrate producing less potent products. Recently a series of novel oxygenated derivatives of omega-3 PUFA that possess potent anti-inflammatory and immunoregulatory actions provided an alternative and perhaps important new role for these essential fatty acids as precursors for potent bioactive protective mediators. The trivial name Resolvin (resolution phase interaction product) was introduced for these bioactive compounds (6, 7). Resolvin E1 (RvE1) is endogenously biosynthesized from EPA in the presence of aspirin during the spontaneous resolution phase of acute inflammation where specific cell-cell interactions occur. Recently organic synthesis was achieved that permitted the complete stereochemical assignment of RvE1 as 5S,12R,18R-trihydroxy-6Z,8E,10E,14Z,16E-eicosapentaenoic acid (8). RvE1 possesses unique counter-regulatory actions that inhibit PMN transendothelial migration in vitro and also act as a potent inhibitor of leukocyte infiltration, dendritic cell migration and IL-12 production in vivo (7, 8).

Inflammatory bowel disease (IBD), including Crohn's disease and ulcerative colitis, is a chronic and relapsing inflammatory disorder characterized by abnormalities in mucosal responses to normally harmless bacterial antigens, abnormal cytokine production, and an inflammatory process associated with mucosal damage (9,10). As such, IBD is characterized by intestinal inflammation associated with leukocytosis and pro-inflammatory gene expression. Results from human studies have suggested that fish oils rich in omega-3 PUFA are protective in reducing the rate of relapse in Crohn's disease (4) and ulcerative colitis (Loeschke D. et. al, Dig. Dis. Sci. 1996, vol 41, 2087-94), but the molecular mechanism underlying this beneficial effect remained to be elucidated.

A need therefore exists for an improved understanding of the function of these materials in physiology as well as the isolation of bioactive agents that can serve to eliminate or diminish various disease states or conditions, such as those associated with gastrointestinal conditions.

### BRIEF SUMMARY OF THE INVENTION

The present invention, in one embodiment, is drawn to isolated therapeutic agents generated from the interaction between a dietary omega-3 polyunsaturated fatty acid (PUFA) such as eicosapentaenoic acid (EPA) or docosahexaenoic acid (DHA), an oxygenase, such as cyclooxygenase-II (COX-2), and an analgesic, such as aspirin (ASA). Surprisingly, careful and challenging isolation of previously unknown and unappreciated compounds are generated from exudates by the combination of components in an appropriate environment to provide di- and tri-hydroxy EPA and DHA derivatives having unique structural and physiological properties. The present invention therefore provides for many new useful therapeutic di- and tri-hydroxy derivatives of EPA or DHA that diminish, prevent, or eliminate gastrointestinal disorders, including colitis, Crohn's disease and irritable bowel syndrome or disease (IBD).

Resolvins, such as resolvin E1 (RvE1; 5S,12R,18R-trihydroxyeicosapentaenoic acid) are novel anti-inflammatory lipid mediators derived from omega-3 fatty acid eicosapentaenoic acid (EPA). At the local site of inflammation, aspirin treatment enhances EPA conversion to 18R-oxygenated products including RvE1 that carry potent anti-inflammatory signals. Surprisingly, resolvins (the compounds identified throughout the specification) such as RvE1 protected against the development of 2,4,6-trinitrobenzene sulfonic acid (TNBS)-induced colitis, a well appreciated experimental colitis model.

The beneficial effect was reflected by increased survival rates, sustained body weight, improvement of histologic scores, reduced serum anti-TNBS IgG, decreased leukocyte infiltration and pro-inflammatory gene expression including IL-12p40, TNF-α, and iNOS. Thus, the novel endogenous lipid mediators termed “resolvins”, such as RvE1 counterregulate leukocyte-mediated tissue injury and pro-inflammatory gene expression. These findings show a novel endogenous mechanism that may underlie the beneficial actions of omega-3 EPA and provides new approaches for the treatment of intestinal inflammation.

The di- and tri-hydroxy EPA and DHA therapeutic agents of the invention useful to treat gastrointestinal disorders include, for example:

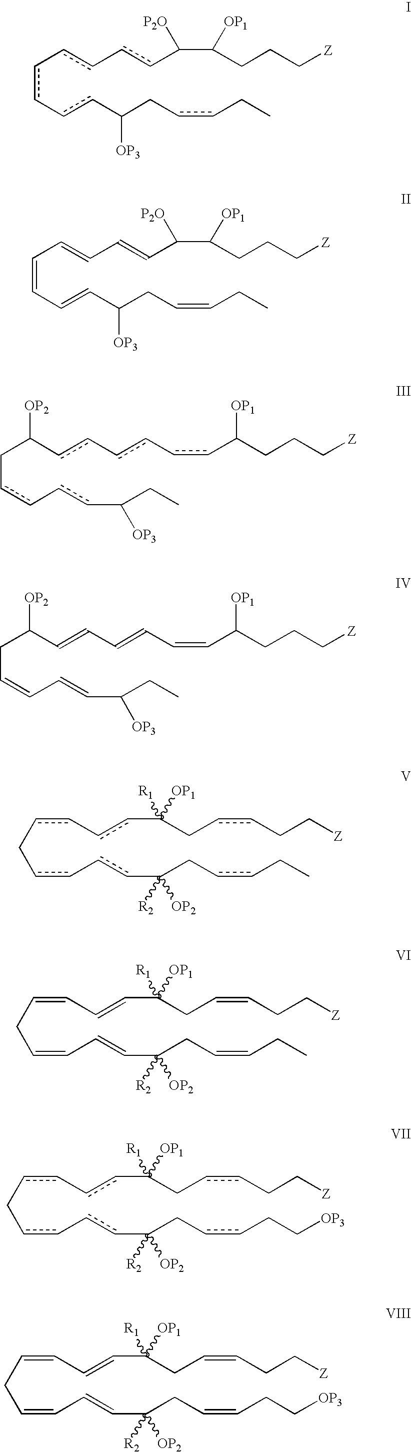

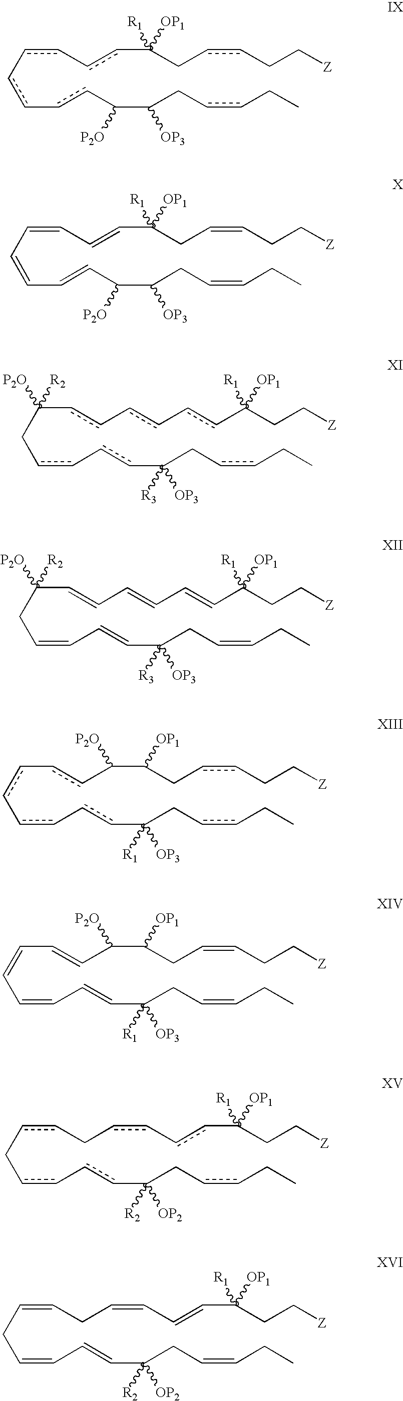

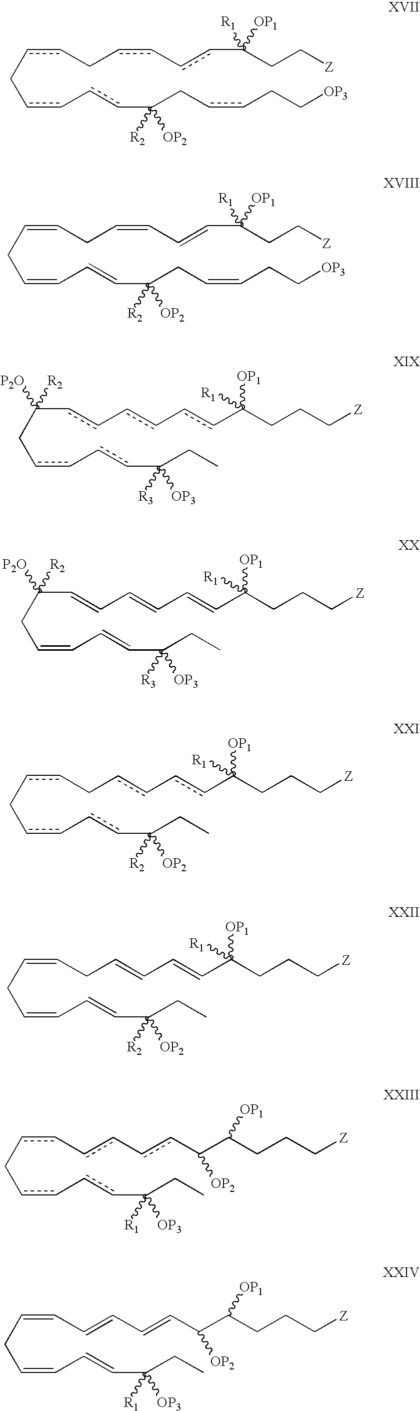

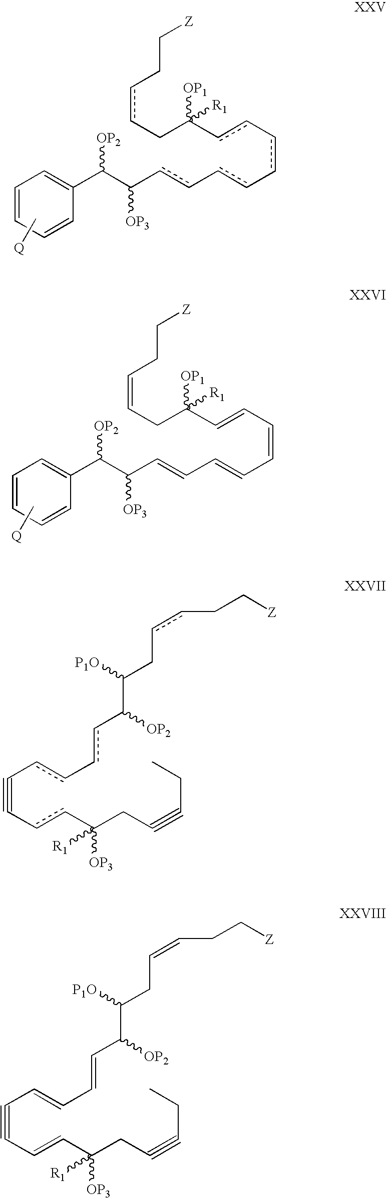

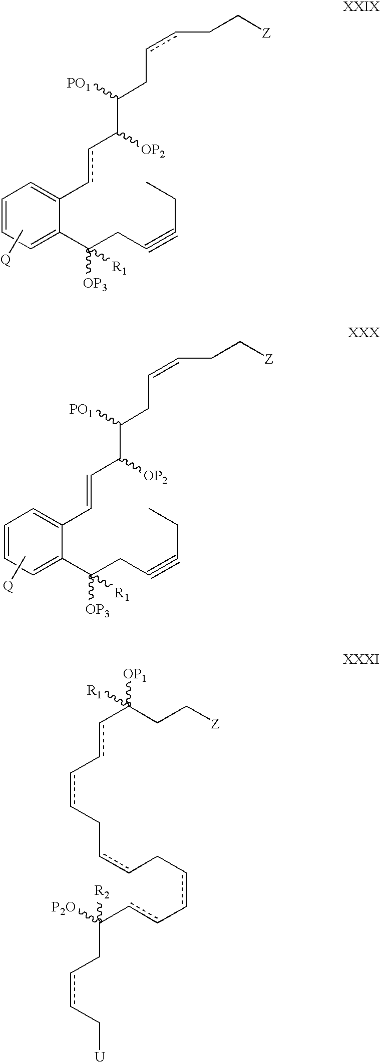

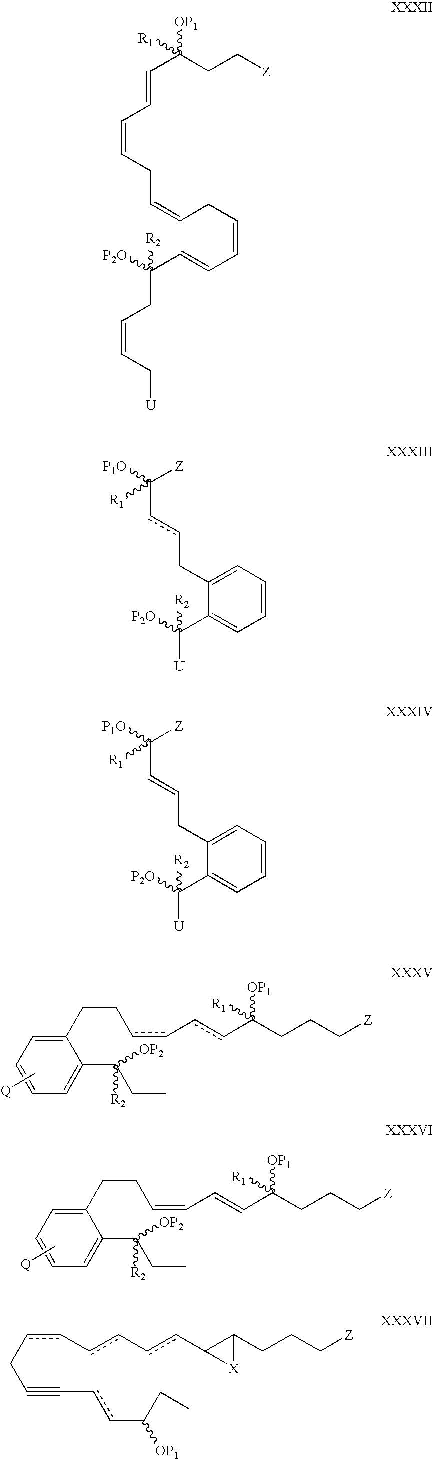

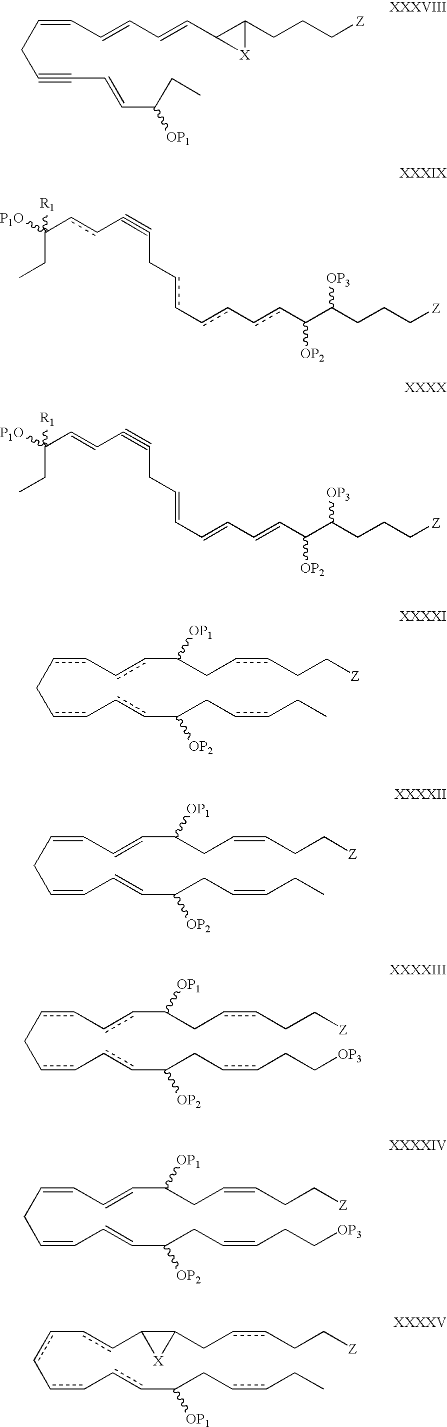

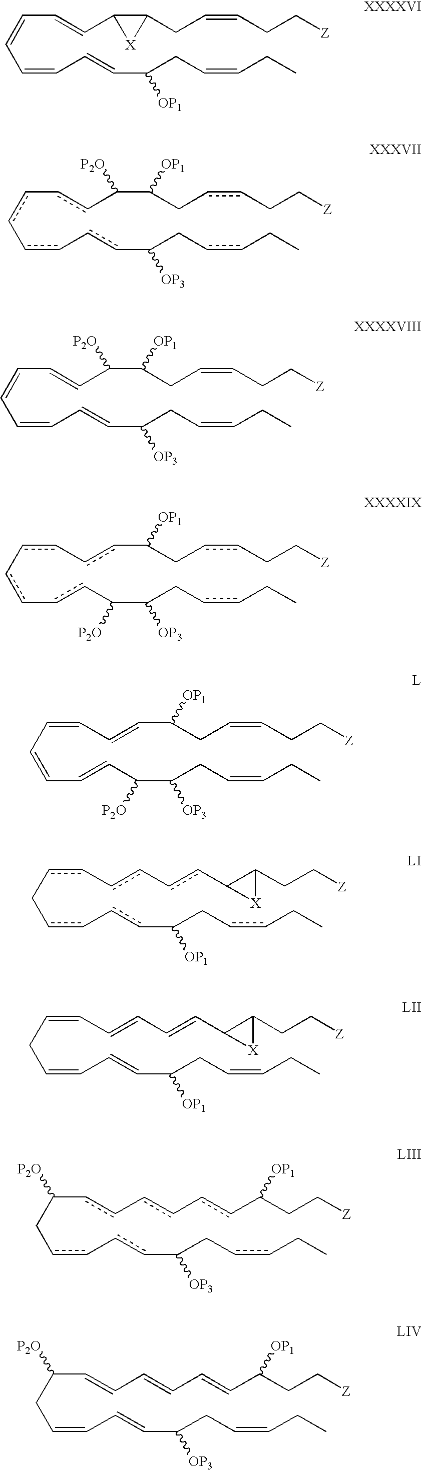

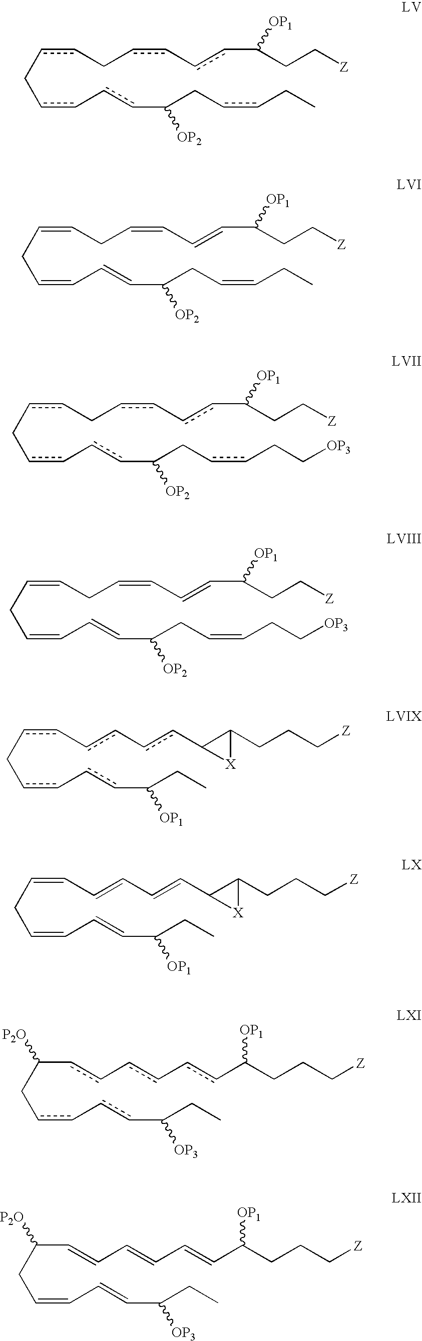

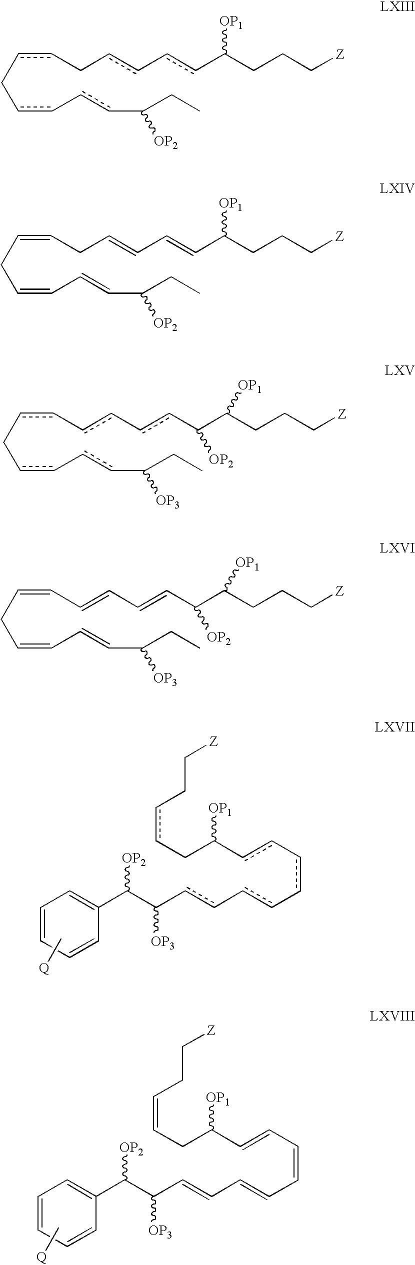

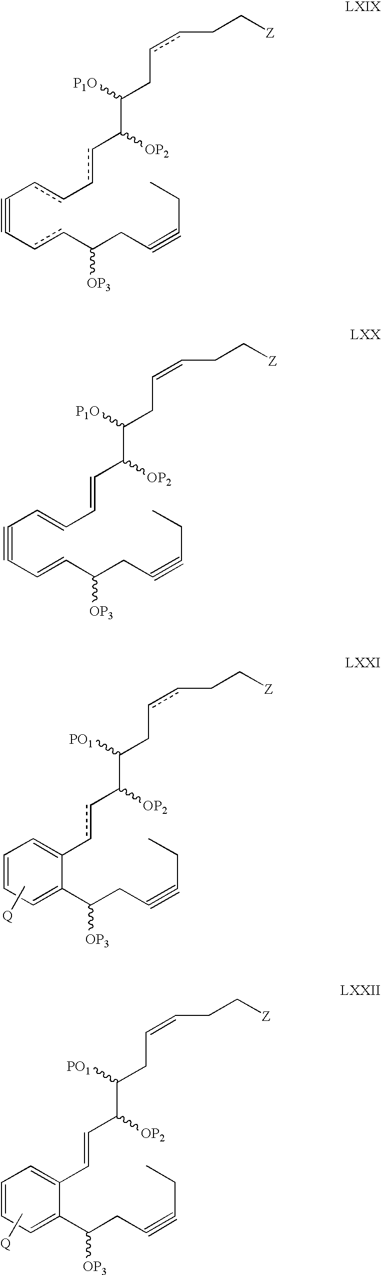

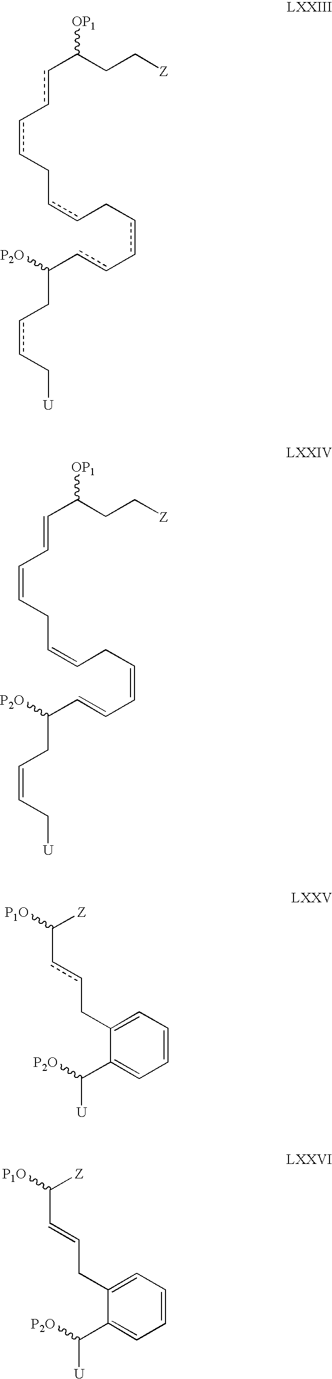

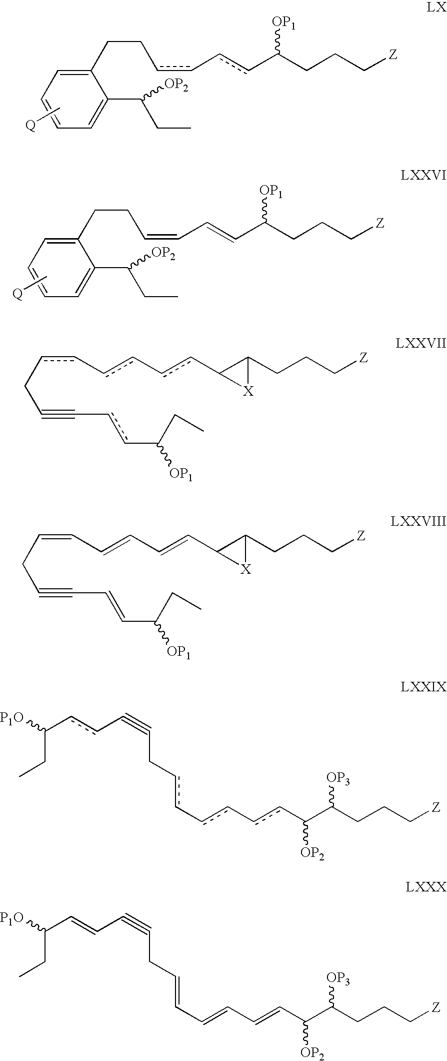

wherein a bond depicted as [symbol] represents either a cis or trans double bond;

wherein P₁, P₂ and P₃, if present, each individually are protecting groups, hydrogen atoms or combinations thereof;

wherein R₁, R₂ and R₃, if present, each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof;

wherein Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;

each R^{a}, if present, is independently selected from the group consisting of hydrogen, (C1-C6)alkyl, (C3-C8)cycloalkyl, cyclohexyl, (C4-C11)cycloalkylalkyl, (C5-C10)aryl, phenyl, (C6-C16)arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each R^{b}, if present, is a suitable group independently selected from the group consisting of ═O, —OR^{d}, (C1-C3)haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]OR^{d}, —[NR^{a}C(O)]OR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};

each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;

each n, independently, if present, is an integer from 0 to 3;

each R^{d}, independently, if present, is a protecting group or R^{a};

in particular, Z is a carboxylic acid, ester, amide, thiocarbamate, carbamate, thioester, thiocarboxamide or a nitrile;

wherein X, if present, is a substituted or unsubstituted methylene, an oxygen atom, a substituted or unsubstituted nitrogen atom, or a sulfur atom;

wherein Q, if present, represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group;

wherein U, if present, is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;

and pharmaceutically acceptable salts thereof.

In certain embodiments, Z is a pharmaceutically acceptable salt of a carboxylic acid, and in particular is an ammonium salt or forms a prodrug.

In certain embodiments, P₁, P₂, and P₃, if present, each individually are hydrogen atoms and Z is a carboxylic acid or ester. In other embodiments, X is an oxygen atom, one or more P's are hydrogen atoms, and Z is a carboxylic acid or ester. In still other embodiments, Q is one or more halogen atoms, one or more P's are hydrogen atoms, and Z is a carboxylic acid or ester.

In certain embodiments, R₁, R₂ and R₃, if present, are each individually lower alkyl groups, such as methyl, ethyl, and propyl and can be halogenated, such as trifluoromethyl. In one aspect, at least one of R₁, R₂ and R₃, if present, is not a hydrogen atom. Generally, Z is a carboxylic acid and one or more P's are hydrogen atoms.

In certain embodiments, when OP₃ is disposed terminally within the resolvin analog, the protecting group can be removed to afford a hydroxyl. Alternatively, in certain embodiments, the designation of OP₃ serves to denote that the terminal carbon is substituted with one or more halogens, i.e., the terminal C-18, C-20, or C-22 carbon, is a trifluoromethyl group, or arylated with an aryl group that can be substituted or unsubstituted as described herein. Such manipulation at the terminal carbon serves to protect the resolvin analog from omega P₄₅₀ metabolism that can lead to biochemical inactivation.

In certain embodiments, P₁, P₂, and P₃, if present, each individually are hydrogen atoms and Z is a carboxylic ester. In other embodiments, P₁, P₂, and P₃, if present, each individually are hydrogen atoms and Z is not carboxylic acid.

In one aspect, the compounds described herein are isolated and/or purified, in particular, compounds in which P₁, P₂, and P₃, if present, each individually are hydrogen atoms and Z is a carboxylic acid, are isolated and or purified.

In certain aspects of the invention, particular compounds are not included; these include the even numbered compounds identified above by Roman numbers, i.e., II, IV, VI, VIII, X, etc. through LXXX.

In one aspect, the resolvins described herein that contain epoxide, cyclopropane, azine, or thioazine rings within the structure also serve as enzyme inhibitors that increase endogenous resolvin levels in vivo and block “pro” inflammatory substances, their formation and action in vivo, such as leukotrienes and/or LTB₄.

Another embodiment of the present invention is directed to pharmaceutical compositions of the novel compounds described throughout the specification useful to treat gastrointestinal conditions.

The present invention also provides methods to treat various disease states and conditions, including for example, gastrointestinal conditions.

The present invention further provides various methods to prepare the novel compounds described throughout the specification.

The present invention also provides packaged pharmaceuticals that contain the novel di- and tri-hydroxy EPA and DHA derivatives described throughout the specification for use in treatment with various gastrointestinal disease states and conditions.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1. (A) Inflammatory exudates from mice treated with EPA and aspirin generate RvE1 in vivo. (B) Inhibition of leukocyte infiltration in zymosan-induced peritonitis treated with EPA and aspirin. Mice were pre-treated for 30 min by aspirin (0.5 mg) intraperitoneally followed by EPA (0.3 mg) and zymosan. Peritoneal lavages were collected at 2 h and cells were enumerated. (n=4, *p<0.05 compared with vehicle control, **p<0.05 compared with aspirin alone). (C) Inhibition of leukocyte infiltration in zymosan-induced peritonitis. RvE1 (100 ng) was injected intravenously into tail veins followed by zymosan A into the peritoneum. Mice were sacrificed, and peritoneal lavages were collected (2 h) and cells enumerated (n=3, *p<0.01 compared with vehicle control).

FIG. 2. RvE1 dramatically attenuates TNBS-induced colitis. (A) The survival rate of RvE1 treated mice was significantly higher than non-treated mice. (B) Wasting disease measured by body weight loss in mice with TNBS colitis is improved by RvE1 treatment. Vehicle control (open diamonds), TNBS only (open circles) and TNBS treated with RvE1 (1 μg/mouse or 0.05 mg/kg) (closed circles). *p<0.01 and **p<0.001 compared with vehicle control, n=6. (C) Macroscopic appearance of the colon of mice receiving vehicle, TNBS only, or mice receiving RvE1 (0.05 mg/kg). (D) Length of the inflamed colons of mice treated with TNBS alone or mice given RvE1. *p<0.01 compared to TNBS alone.

FIG. 3. Colon histopathology from RvE1 or ATLa-treated mice. Colon section of vehicle-treated mouse (A), 4 days after the induction of colitis by TNBS (B), mice received RvE1 (C) and ATLa (D). (E) Histological scores of the colon of mice receiving vehicle, TNBS only, TNBS plus RvE1, or TNBS plus ATLa. *p<0.05 and **p<0.01, respectively.

FIG. 4. RvE1 reduces leukocyte infiltration and pro-inflammatory mediators in colitis. (A) Myeloperoxidase (MPO) activity of colons of mice treated with TNBS alone or mice given RvE1. *p<0.0001 versus mice treated with TNBS alone. (B) Anti-TNBS IgG level in serum on day 4 from mice treated with TNBS alone or receiving RvE1 as noted. *p<0.01. (C) Quantitative real time PCR analysis of mRNA expression of inflammatory mediators in colons obtained on day 4 from control mice (white column), mice treated with TNBS alone (black column) or receiving TNBS plus RvE1 (gray column). *p<0.01.

FIG. 5. ChemR23 mRNA is expressed in mouse colon. Total RNA was isolated from control and TNBS-treated colons and RT-PCR was performed as described in Methods.

### DETAILED DESCRIPTION

The present invention provides methods for preparing novel anti-inflammatory agents and discloses the structures of novel endogenously generated anti-inflammatory mediators that are generated in resolution. The invention is based on the structural elucidation of several new classes of compounds that are generated in vivo during inflammation, which are termed “resolvins.” The structural elucidation of the compounds and the mechanisms of their biosynthesis at sites of inflammation in vivo in murine systems via vascular leukocyte interactions and in brain when aspirin is taken are presented throughout the specification. This structural elucidation of novel biochemical pathways and compounds that serve as endogenous mediators in anti-inflammation and/or pro-resolution forms the basis of a novel approach to active anti-inflammatories that expedites resolution.

From structural elucidation, these novel compounds are “active ingredients” that the body converts via novel biochemical pathways to endogenous omega-3 fatty acid-derived mediators that have anti-inflammatory properties that we've uncovered in murine models. These results provide that these compounds, when generated in vivo in humans, are responsible, at least in part, for the beneficial actions of eating fish and aspirin therapy.

The structural elucidation of these pathways, biological properties and structural elucidation of novel compounds formulates the basis for a novel therapeutic approach, namely administering these compounds and/or related structures/analogs with greater biostability and chemical stability as new therapeutic approaches to expedite resolution and evoke anti-inflammation status.

Along these lines, the new structures, pathways, and examples of novel chemical classes of analogs based on these natural resolvin compounds are presented in the illustrations and figures throughout this specification. Most importantly, with the description of these novel pathways and physical properties of the resolving, one claim can be directed for assaying these compounds in human fluids (blood, urine, breast milk), biopsied material, etc. as treatment markers to gauge effective n-3 status levels as indices for developing a therapeutic basis for anti-inflammation. This includes LC-MS-MS and GC-MS properties and could also lead to the development of much easier to handle ELISA assays monitoring these novel products.

The present invention is drawn to methods for treating or preventing a gastrointestinal disease or condition in a subject by administration of a combination of a polyunsaturated fatty acid(s) (PUFA(s)) and aspirin, i.e., polyunsaturated fatty acids including C20:5 and C22:6. In one embodiment, the omega fatty acid, e.g., C20:3 or C22:6, and an analgesic, such as aspirin, are administered at two different times.

The phrase “resolvin mediated interaction” is intended to include disease states or conditions caused by or associated with one or more types of inflammation associated with cytokine, leukocyte or PMN regulation and regulation by one or more of the therapeutic analogs described throughout the specification for the pharmacologic inhibition of gastrointestinal condition(s). In one embodiment, the disease state includes, for example, those diseases that afflict a subject by associating with or interfering with cytokine, leukocyte or PMN regulation within the subject. Such disease states or conditions are described throughout the specification, vide infra, and are incorporated herein in their entirety. Presently unknown conditions related to cytokine, leukocyte or PMN regulation that may be discovered in the future are encompassed by the present invention, since the characterization as conditions related to resolvin mediated interaction(s) will be readily determinable by persons skilled in the art.

Resolvins are natural counter regulatory lipid mediators in host defense mechanisms that protect host tissues from effector cell mediated injury and over amplification of acute inflammation to dampen the inflammatory response, i.e., counterregulative. Some known chronic inflammatory diseases may represent the loss of and/or genetically program low resolvin edogenous responders and/or levels. The resolvin analogs described throughout the specification can be used to replace, enhance and/or treat the loss of these substances therapeutically and thereby pharmacologically resolve inflammation by inhibiting leukocyte recruitment and amplification, namely inhibition of the amplification of inflammation.

The present invention is also drawn to methods for treating disease states or conditions that are associated with inflammation (hence “resolving”), the recruitment of neutrophils, leukocytes and/or cytokines are included within the general scope of gastrointestinal inflammation.

The phrase “gastrointestinal disease” or “gastrointestinal condition” includes many gastrointestinal inflammatory disorders of the digestive system (mouth, stomach, esophagus, small intestine and large intestine), including but not limited to, stomatitis, periodontitis, esophagitis, gastritis, inflammatory bowel diseases such as ulcerative colitis and Crohn's disease, infectious enteritis (viral, bacterial, parasitic), antibiotic associative diarrhea, clostridium difficile colitis, microscopic or lymphocytic colitis, collagenous colitis, colon polyps and familial polyp syndromes (e.g., familial polyposis syndrome, Gardner's Syndrome), helicobacter pylori, irritable bowel syndrome, nonspecific diarrheal illnesses, and intestinal cancers.

The phrase “gastrointestinal inflammation” refers to inflammation of a mucosal layer of the gastrointestinal tract, and encompasses acute and chronic inflammatory conditions. Acute inflammation is generally characterized by a short time of onset and infiltration or influx of neutrophils. Chronic inflammation is generally characterized by a relatively longer period of onset and infiltration or influx of mononuclear cells. Chronic inflammation can also typically characterized by periods of spontaneous remission and spontaneous occurrence.

The phrase “mucosal layer of the gastrointestinal tract” is meant to include mucosa of the bowel (including the small intestine and large intestine), rectum, stomach (gastric) lining, oral cavity, and the like.

The phrase “chronic gastrointestinal inflammation” refers to inflammation of the mucosal of the gastrointestinal tract that is characterized by a relatively longer period of onset, is long-lasting (e.g., from several days, weeks, months, or years and up to the life of the subject), and is associated with infiltration or influx of mononuclear cells and can be further associated with periods of spontaneous remission and spontaneous occurrence. Subjects with chronic gastrointestinal inflammation may be expected to require a long period of supervision, observation, or care.

The phrase “chronic gastrointestinal inflammatory conditions” (also referred to as “chronic gastrointestinal inflammatory diseases”) having such chronic inflammation include, but are not necessarily limited to, inflammatory bowel disease (IBD), colitis induced by environmental insults (e.g., gastrointestinal inflammation (e.g., colitis) caused by or associated with (e.g., as a side effect) a therapeutic regimen, such as administration of chemotherapy, radiation therapy, and the like), colitis in conditions such as chronic granulomatous disease, celiac disease, celiac sprue (a heritable disease in which the intestinal lining is inflamed in response to the ingestion of a protein known as gluten), food allergies, gastritis, infectious gastritis or enterocolitis (e.g., Helicobacter pylori-infected chronic active gastritis) and other forms of gastrointestinal inflammation caused by an infectious agent, and other like conditions.

The phrase “inflammatory bowel disease” or “IBD” refers to any of a variety of diseases characterized by inflammation of all or part of the intestines. Examples of inflammatory bowel disease include, but are not limited to, Crohn's disease and ulcerative colitis. Reference to IBD throughout the specification is often referred to in the specification as exemplary of gastrointestinal inflammatory conditions, and is not meant to be limiting.

Terms and abbreviations used throughout the specification include:
ASA, aspirin
ATLa, aspirin triggered lipoxin A₄
COX, cyclooxygenase
EPA, eicosapentaenoic acid
DHA, docosahexaenoic acid
DSS, dextran sodium sulfate
GC-MS, gas chromatography-mass spectrometry
7S,17R-dihydroxy-DHA, 7S,17R-dihydroxy-docosa-4Z,8E,10Z,13Z,15E,19Z-hexaenoic acid
4S,17R-dihydroxy-DHA, 4S-17R-dihydroxy-docosa-5E,7Z,10Z,13Z,15E,19Z-hexaenoic acid
7S,17R,22-trihydroxy-DHA, 7S,17R,22-trihydroxy-docosa-4Z,8Z,10Z,13Z,15E,19Z-hexaenoic acid
4S,11,17R-trihydroxy-DHA, 4S,11,17S,-trihydroxy-docosa-5E,7E,9Z,13Z,15E,19Z-hexaenoic acid
IBD, inflammatory bowel disease
LC-UV-MS-MS, liquid chromatography-UV diode array detector-tandem mass spectrometry
LO, lipoxygenase
LT, leukotriene
LX, lipoxins
MPO, myeloperoxidase
PDA, photodiode array detector
PUFA, polyunsaturated fatty acids
RvE1, Resolvin E1, 5S,12R,18R-trihydroxyeicosapentaenoic acid
TNBS, 2,4,6-trinitrobenzene sulfonic acid

“Alkyl” by itself or as part of another substituent refers to a saturated or unsaturated branched, straight-chain or cyclic monovalent hydrocarbon radical having the stated number of carbon atoms (i.e., C1-C6 means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl,” “alkenyl” and/or “alkynyl” is used, as defined below. In preferred embodiments, the alkyl groups are (C1-C6)alkyl.

“Alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. In preferred embodiments, the alkanyl groups are (C1-C6)alkanyl.

“Alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl ; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In preferred embodiments, the alkenyl group is (C2-C6)alkenyl.

“Alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In preferred embodiments, the alkynyl group is (C2-C6)alkynyl.

“Alkyldiyl” by itself or as part of another substituent refers to a saturated or unsaturated, branched, straight-chain or cyclic divalent hydrocarbon group having the stated number of carbon atoms (i.e., C1-C6 means from one to six carbon atoms) derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In preferred embodiments, the alkyldiyl group is (C1-C6)alkyldiyl. Also preferred are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkyleno group is (C1-C6) or (C1-C3)alkyleno. Also preferred are straight-chain saturated alkano groups, e.g., methano, ethano, propano, butano, and the like.

“Heteroalkyl,” Heteroalkanyl,” Heteroalkenyl,” Heteroalkynyl,” Heteroalkyldiyl” and “Heteroalkyleno” by themselves or as part of another substituent refer to alkyl, alkanyl, alkenyl, alkynyl, alkyldiyl and alkyleno groups, respectively, in which one or more of the carbon atoms are each independently replaced with the same or different heteratoms or heteroatomic groups. Typical heteroatoms and/or heteroatomic groups which can replace the carbon atoms include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C1-C6)alkyl.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Acyclic Heteroatomic Bridge” refers to a divalent bridge in which the backbone atoms are exclusively heteroatoms and/or heteroatomic groups. Typical acyclic heteroatomic bridges include, but are not limited to, —O—, —S—, —S—O—, —NR′—, —PH—, —S(O)—, —S(O)₂—, —S(O) NR′—, —S(O)₂NR′—, and the like, including combinations thereof, where each R′ is independently hydrogen or (C1-C6)alkyl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, tetrahydronaphthalene, triphenylene, trinaphthalene, and the like, as well as the various hydro isomers thereof.

“Aryl” by itself or as part of another substituent refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In preferred embodiments, the aryl group is (C5-C15)aryl, with (C5-C10) being even more preferred. Particularly preferred aryls are cyclopentadienyl, phenyl and naphthyl.

“Arylaryl” by itself or as part of another substituent refers to a monovalent hydrocarbon group derived by the removal of one hydrogen atom from a single carbon atom of a ring system in which two or more identical or non-identical parent aromatic ring systems are joined directly together by a single bond, where the number of such direct ring junctions is one less than the number of parent aromatic ring systems involved. Typical arylaryl groups include, but are not limited to, biphenyl, triphenyl, phenyl-naphthyl, binaphthyl, biphenyl-naphthyl, and the like. Where the number of carbon atoms in an arylaryl group are specified, the numbers refer to the carbon atoms comprising each parent aromatic ring. For example, (C5-C15)arylaryl is an arylaryl group in which each aromatic ring comprises from 5 to 15 carbons, e.g., biphenyl, triphenyl, binaphthyl, phenylnaphthyl, etc. Preferably, each parent aromatic ring system of an arylaryl group is independently a (C5-C15) aromatic, more preferably a (C5-C10) aromatic. Also preferred are arylaryl groups in which all of the parent aromatic ring systems are identical, e.g., biphenyl, triphenyl, binaphthyl, trinaphthyl, etc.

“Biaryl” by itself or as part of another substituent refers to an arylaryl group having two identical parent aromatic systems joined directly together by a single bond. Typical biaryl groups include, but are not limited to, biphenyl, binaphthyl, bianthracyl, and the like. Preferably, the aromatic ring systems are (C5-C15) aromatic rings, more preferably (C5-C10) aromatic rings. A particularly preferred biaryl group is biphenyl.

“Arylalkyl” by itself or as part of another substituent refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In preferred embodiments, the arylalkyl group is (C6-C21)arylalkyl, e.g., the alkanyl, alkenyl or alkynyl moiety of the arylalkyl group is (C1-C6) and the aryl moiety is (C5-C15). In particularly preferred embodiments the arylalkyl group is (C6-C13), e.g., the alkanyl, alkenyl or alkynyl moiety of the arylalkyl group is (C1-C3) and the aryl moiety is (C5-C10).

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, S(O), S(O)₂, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include common substituents, such as, for example, benzopyrone and 1-methyl-1,2,3,4-tetrazole. Typical parent heteroaromatic ring systems include, but are not limited to, acridine, benzimidazole, benzisoxazole, benzodioxan, benzodioxole, benzofuran, benzopyrone, benzothiadiazole, benzothiazole, benzotriazole, benzoxaxine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In preferred embodiments, the heteroaryl group is a 5-14 membered heteroaryl, with 5-10 membered heteroaryl being particularly preferred.

“Heteroaryl-Heteroaryl” by itself or as part of another substituent refers to a monovalent heteroaromatic group derived by the removal of one hydrogen atom from a single atom of a ring system in which two or more identical or non-identical parent heteroaromatic ring systems are joined directly together by a single bond, where the number of such direct ring junctions is one less than the number of parent heteroaromatic ring systems involved. Typical heteroaryl-heteroaryl groups include, but are not limited to, bipyridyl, tripyridyl, pyridylpurinyl, bipurinyl, etc. Where the number of atoms are specified, the numbers refer to the number of atoms comprising each parent heteroaromatic ring systems. For example, 5-15 membered heteroaryl-heteroaryl is a heteroaryl-heteroaryl group in which each parent heteroaromatic ring system comprises from 5 to 15 atoms, e.g., bipyridyl, tripuridyl, etc. Preferably, each parent heteroaromatic ring system is independently a 5-15 membered heteroaromatic, more preferably a 5-10 membered heteroaromatic. Also preferred are heteroaryl-heteroaryl groups in which all of the parent heteroaromatic ring systems are identical.

“Biheteroaryl” by itself or as part of another substituent refers to a heteroaryl-heteroaryl group having two identical parent heteroaromatic ring systems joined directly together by a single bond. Typical biheteroaryl groups include, but are not limited to, bipyridyl, bipurinyl, biquinolinyl, and the like. Preferably, the heteroaromatic ring systems are 5-15 membered heteroaromatic rings, more preferably 5-10 membered heteroaromatic rings.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In preferred embodiments, the heteroarylalkyl group is a 6-21 membered heteroarylalkyl, e.g., the alkanyl, alkenyl or alkynyl moiety of the heteroarylalkyl is (C1-C6)alkyl and the heteroaryl moiety is a 5-15-membered heteroaryl. In particularly preferred embodiments, the heteroarylalkyl is a 6-13 membered heteroarylalkyl, e.g., the alkanyl, alkenyl or alkynyl moiety is (C1-C3)alkyl and the heteroaryl moiety is a 5-10 membered heteroaryl.

“Halogen” or “Halo” by themselves or as part of another substituent, unless otherwise stated, refer to fluoro, chloro, bromo and iodo.

“Haloalkyl” by itself or as part of another substituent refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C1-C2)haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

The above-defined groups may include prefixes and/or suffixes that are commonly used in the art to create additional well-recognized substituent groups. As examples, “alkyloxy” or “alkoxy” refers to a group of the formula —OR″, “alkylamine” refers to a group of the formula —NHR″ and “dialkylamine” refers to a group of the formula —NR″R″, where each R″ is independently an alkyl. As another example, “haloalkoxy” or “haloalkyloxy” refers to a group of the formula —OR′″, where R′″ is a haloalkyl.

“Protecting group” refers to a group of atoms that, when attached to a reactive functional group in a molecule, mask, reduce or prevent the reactivity of the finctional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPPS groups), glycol ethers, such as ethylene glycol and propylene glycol derivatives and allyl ethers.

Throughout the following descriptions, it should be understood that where particular double bonding is depicted, it is intended to include both cis and trans configurations. Exemplary formulae are provided with specific configurations, but for completeness, the double bonds can be varied. Not every structural isomer is shown in efforts to maintain brevity of the specification. However, this should not be considered limiting in nature. Additionally, where synthetic schemes are provided, it should be understood that all cis/trans configurational isomers are also contemplated and are within the scope and purvue of the synthesis. Again, particular double bonding is depicted in exemplary manner.

The present invention provides compounds and pharmaceutical compositions useful for the treatment of gastrointestinal conditions, having the formula:

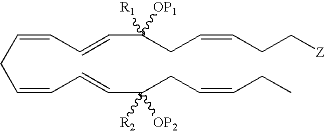

P₁ and P₂ each individually are protecting groups, hydrogen atoms or combinations thereof.

R₁ and R₂ each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof.

Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;

each R^{a}, if present, is independently selected from the group consisting of hydrogen, (C1-C6)alkyl, (C3-C8)cycloalkyl, cyclohexyl, (C4-C11)cycloalkylalkyl, (C5-C10)aryl, phenyl, (C6-C16)arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;

each R^{b}, if present, is a suitable group independently selected from the group consisting of ═O, —OR^{d}, (C1-C3)haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(a)NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R and —[NR^{a}C(NR^{a})]ₙNR^{c}R^{c};

each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;

each n, independently, if present, is an integer from 0 to 3;

each R^{d}, independently, if present, is a protecting group or R^{a};

and pharmaceutically acceptable salts thereof.

In certain embodiments, P₁ and P₂ are hydrogen atoms, R₁ and R₂ each individually are methyl groups or hydrogen atoms or combinations thereof, and Z is carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of gastrointestinal conditions, having the formula:

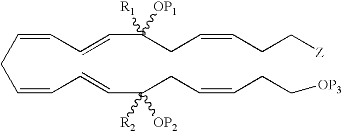

P₁, P₂ and P₃ each individually are protecting groups, hydrogen atoms or combinations thereof and R₁, R₂ and Z are as defined above.

In certain embodiments, P₁, P₂ and P₃ each are hydrogen atoms, R₁ and R₂ each individually are methyl groups or hydrogen atoms or combinations thereof and Z is a carboxylic acid or a carboxylic ester.

In certain aspects the designation of OP₃ serves to denote that the terminal carbon is substituted with one or more halogens (I, Cl, F, Br, mono, di or tri substitution) to form, for example, a trifluoromethyl group, or is an aryl group or phenoxy group that can be substituted or unsubstituted as described herein.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of gastrointestinal conditions, having the formula:

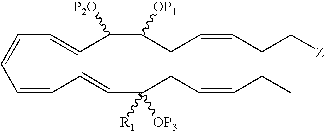

P₁, P₂, P₃, R₁ and Z are as defined above.

In an embodiment, P₁, P₂ and P₃ each are hydrogen atoms, R₁ is a methyl group or a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of gastrointestinal conditions, having the formula:

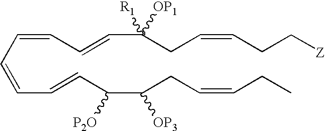

P₁, P₂, P₃, R₁ and Z are as defined above.

In a particular embodiment, P₁, P₂ and P₃ each are hydrogen atoms, R₁ is a methyl group or a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of gastrointestinal conditions, having the formula:

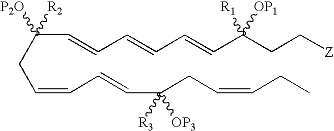

P₁, P₂, P₃ and Z are as defined above.

R₁, R₂ and R₃, each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof.

In a particular embodiment, P₁, P₂ and P₃ each are hydrogen atoms, R₁, R₂ and R₃ are each hydrogen atoms and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention also provides compounds and pharmaceutical compositions useful for the treatment of gastrointestinal conditions, having the formula:

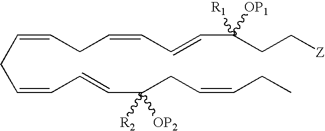

P₁, P₂, R₁, R₂ and Z are as defined above.

In certain embodiments, P₁ and P₂ are hydrogen atoms, R₁ and R₂ each individually are methyl groups or hydrogen atoms or combinations thereof and Z is carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

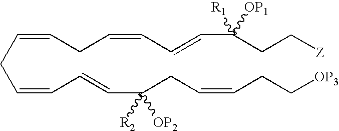

P₁, P₂, P₃, R₁, R₂ and Z are as defined above.

In a particular embodiment, P₁, P₂ and P₃ each are hydrogen atoms and Z is a carboxylic acid or a carboxylic ester.

In certain aspects the designation of OP₃ serves to denote that the terminal carbon is substituted with one or more halogens (I, Cl, F, Br, mono, di or tri substitution) to form, for example, a trifluoromethyl group, or is an aryl group or phenoxy group that can be substituted or unsubstituted as described herein.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of gastrointestinal conditions, having the formula:

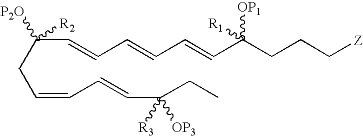

P₁, P₂, P₃, R₁, R₂, R₃ and Z are as defined above.

In one embodiment, P₁, P₂ and P₃ each are hydrogen atoms, R₁, R₂ and R₃ each individually are methyl groups or hydrogen atoms or combinations thereof and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

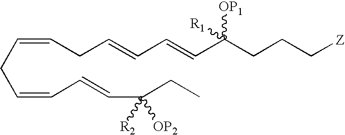

P₁, P₂, R₁, R₂ and are as defined above.

In one particular embodiment, P₁ and P₂ are hydrogen atoms, R₁ and R₂ each individually are methyl groups or hydrogen atoms or combinations thereof and Z is carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

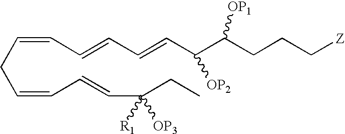

P₁, P₂, P₃, R₁ and Z are as defined above.

In one embodiment, P₁, P₂ and P₃ each are hydrogen atoms, R₁ is a methyl group or a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

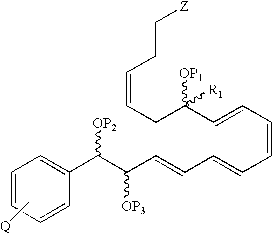

P₁, P₂, P₃, R₁ and Z are as defined above.

Q represents one or more substituents and each Q, independently, is a hydrogen atom, a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group.

In one particular aspect, P₁, P₂ and P₃ each are hydrogen atoms, R₁ is a methyl group or a hydrogen atom, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

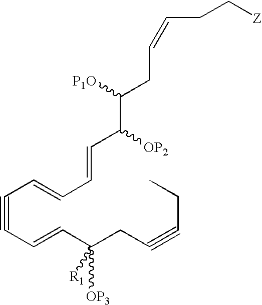

P₁, P₂, P₃, R₁ and Z are as defined above.

In one embodiment, P₁, P₂ and P₃ each are hydrogen atoms, R₁ is a methyl group or a hydrogen atom, and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention also provides compounds and pharmaceutical compositions useful for the treatment of gastrointestinal conditions, having the formula:

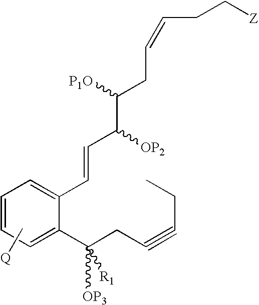

P₁, P₂, P₃, R₁, Q and Z are as defined above.

In one particular embodiment, P₁, P₂ and P₃ each are hydrogen atoms, R₁ is a methyl group or a hydrogen atom, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

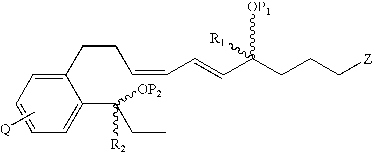

P₁, P₂, R₁, R₂, Q and Z are as defined above.

In one embodiment, P₁, and P₂ each are hydrogen atoms, R₁ and R₂ each individually are methyl groups or hydrogen atoms or combinations thereof, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

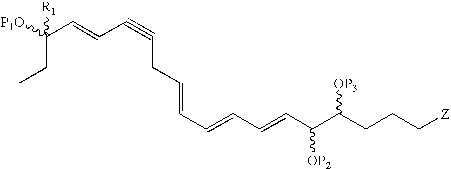

P₁, P₂, P₃, R₁ and Z are as defined above.

In one embodiment, P₁, P₂ and P₃ each are hydrogen atoms, R₁ is a methyl group or a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

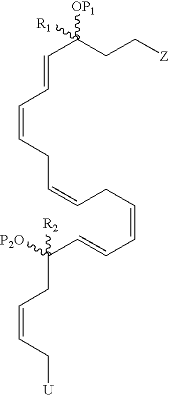

P₁, P₂, R₁, R₂ and Z are as defined above.

U is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group.

In one aspect, P₁, and P₂ each are hydrogen atoms, R₁ and R₂ each individually are methyl groups or hydrogen atoms or combinations thereof, U is a trifluoromethyl group and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention still further provides compounds and pharmaceutical compositions useful for the treatment of gastrointestinal conditions, having the formula:

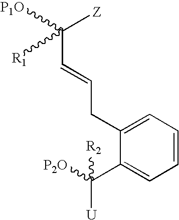

P₁, P₂, R₁, R₂, U and Z are as defined above.

For example, P₁, and P₂ each are hydrogen atoms, R₁ and R₂ each individually are methyl groups or hydrogen atoms or combinations thereof, U is a trifluoromethyl group and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of gastrointestinal conditions, having the formula:

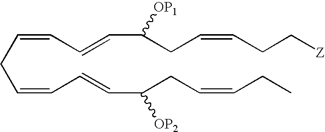

P₁, P₂ and Z are as defined above.

In certain embodiments, P₁ and P₂ are hydrogen atoms and Z is carboxylic acid or a carboxylic ester. In certain embodiments, when P₁ and P₂ are hydrogen atoms and Z is a carboxylic acid, the compound is either isolated and/or purified.

Purity of such compounds is at least 80% pure, in particular, greater than 90% pure, more particularly greater than 95% pure and most particularly greater than about 99% pure, based on analytical measurements such has GC, MS, or ¹HNMR, etc. This applies to all isolated and/or purified compounds throughout the specification.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The analogs are designated as 7,17-diHDHAs. In certain aspects, the chiral carbon atom at the 7 position (C-7) has an R configuration. In another aspect, the C-7 carbon atom preferably has an S configuration. In still another aspect, the C-7 carbon atom is as an R/S racemate. Additionally, the chiral carbon atom at the 17 position (C-17) can have an R configuration. Alternatively, the C-17 carbon can have an S configuration. In still yet another aspect, the C-17 carbon can preferably exist as an R/S racemate. Exemplary analogs include, for example, 7S,17R/S-diHDHA, 7S,17R/S-dihydroxy-docosa-4Z,8E,10Z,13Z,15E,19Z-hexaenoic acid.

The present invention further provides compounds and pharmaceutical compositions useful for the treatment of gastrointestinal conditions, having the formula:

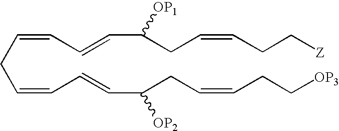

P₁, P₂, P₃ and Z are as defined above.

In certain embodiments, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

In certain aspects the designation of OP₃ serves to denote that the terminal carbon is substituted with one or more halogens (I, Cl, F, Br, mono, di or tri substitution) to form, for example, a trifluoromethyl group, or is an aryl group or phenoxy group that can be substituted or unsubstituted as described herein.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

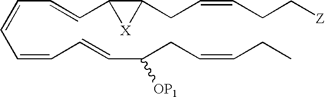

P₁ and Z are as defined above.

X is a substituted or unsubstituted methylene, an oxygen atom, a substituted or unsubstituted nitrogen atom, or a sulfur atom.

In one embodiment, P₁ is a hydrogen atom, X is an oxygen atom and Z is a carboxylic acid or a carboxylic ester. In certain embodiments, when P₁ is a hydrogen atom and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

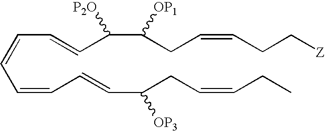

P₁, P₂, P₃ and Z are as defined above.

The analogs are designated as 7,8,17-trihydroxy-DHAs. In certain embodiments, the chiral carbon atom at the 7 position (C-7) has an R configuration. In other embodiments, the C-7 carbon atom preferably has an S configuration. In still other embodiments, the C-7 carbon atom is as an R/S racemate. In certain aspects, the chiral carbon atom at the 8 position (C-8) has an R configuration. In another aspect, the C-8 carbon atom has an S configuration. In still another aspect, the C-8 carbon atom preferably is as an R/S racemate. Additionally, the chiral carbon atom at the 17 position (C-17) can have an R configuration. Alternatively, the C-17 carbon can preferably have an S configuration. In still yet another aspect, the C-17 carbon can exist as an R/S racemate.

In a particular embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

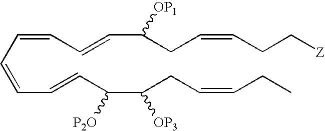

P₁, P₂, P₃ and Z are as defined above.

In a particular embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

The analogs are designated as 7,16,17-trihydroxy-DHAs. In certain embodiments, the chiral carbon atom at the 7 position (C-7) has an R configuration. In other embodiments, the C-7 carbon atom preferably has an S configuration. In still other embodiments, the C-7 carbon atom is as an R/S racemate. In certain aspects, the chiral carbon atom at the 16 position (C-16) has an R configuration. In another aspect, the C-16 carbon atom has an S configuration. In still another aspect, the C-16 carbon atom preferably is as an R/S racemate. Additionally, the chiral carbon atom at the 17 position (C-17) can have an R configuration. Alternatively, the C-17 carbon can preferably have an S configuration. In still yet another aspect, the C-17 carbon can exist as an R/S racemate.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

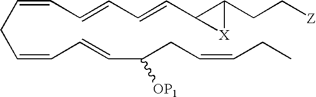

P₁, X and Z are as defined above.

In one embodiment, P₁ is a hydrogen atom, X is an oxygen atom and Z is a carboxylic acid or a carboxylic ester. In certain embodiments, when P₁ is a hydrogen atom and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

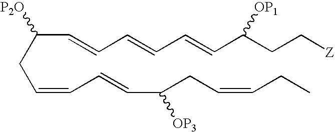

P₁, P₂, P₃ and Z are as defined above.

In a particular embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

The analogs are designated as 4,11,17-trihydroxy-DHAs. In certain embodiments, the chiral carbon atom at the 4 position (C-4) has an R configuration. In other embodiments, the C-4 carbon atom preferably has an S configuration. In still other embodiments, the C-4 carbon atom is as an R/S racemate. In certain aspects, the chiral carbon atom at the 11 position (C-11) has an R configuration. In another aspect, the C-11 carbon atom has an S configuration. In still another aspect, the C-11 carbon atom preferably is as an R/S racemate. Additionally, the chiral carbon atom at the 17 position (C-17) can have an R configuration. Alternatively, the C-17 carbon can preferably have an S configuration. In still yet another aspect, the C-17 carbon can exist as an R/S racemate.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of gastrointestinal conditions, having the formula:

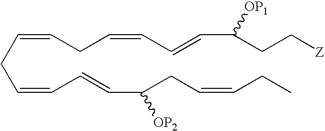

P₁, P₂ and Z are as defined above.

In certain embodiments, P₁ and P₂ are hydrogen atoms and Z is carboxylic acid or a carboxylic ester. In certain embodiments, when P₁, and P₂ are hydrogen atoms and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

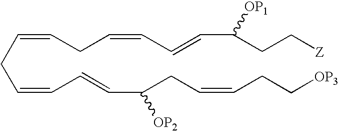

P₁, P₂, P₃ and Z are as defined above.

In a particular embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

In certain aspects the designation of OP₃ serves to denote that the terminal carbon is substituted with one or more halogens (I, Cl, F, Br, mono, di or tri substitution) to form, for example, a trifluoromethyl group, or is an aryl group or phenoxy group that can be substituted or unsubstituted as described herein.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

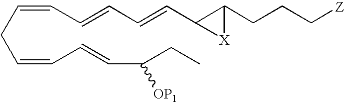

P₁, X and Z are as defined above.

In one embodiment P₁ is a hydrogen atom, X is an oxygen atom and Z is a carboxylic acid or a carboxylic ester. In certain embodiments, when P₁ is a hydrogen atom and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of gastrointestinal conditions, having the formula:

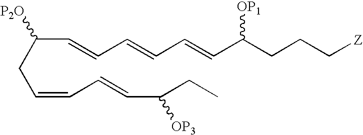

P₁, P₂, P₃ and Z are as defined above.

In one embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention provides compounds and pharmaceutical compositions useful for the treatment of gastrointestinal conditions, having the formula:

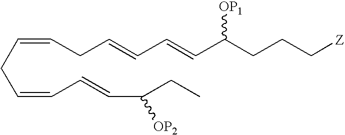

P₁, P₂ and Z are as defined above.

In one particular embodiment, P₁ and P₂ are hydrogen atoms and Z is carboxylic acid or a carboxylic ester. In certain embodiments, when P₁ and P₂ are hydrogen atoms and Z is a carboxylic acid, the compound is either isolated and/or purified.

In one particular embodiment, the compound is 5S,18(+/−)-diHEPA, wherein the carboxyl group can be an acid, ester or salt. 5S,18(+/−)-diHEPA has been synthesized with 5-lipoxygenase potato and its physical properties are based on LC-MS-MS. Additionally, 5S,18(+/−)-diHEPA is biologically active and has anti-inflammatory activity as noted by the downregulation of PMN infiltration in a peritonitis model. It is equipotent to Resolvin E1 at an equal dose amount.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

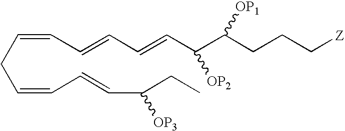

P₁, P₂, P₃ and Z are as defined above.

In one embodiment, P₁, P₂ and P₃ each are hydrogen atoms and Z is a carboxylic acid or a carboxylic ester. In certain embodiments, when P₁, P₂ and P₃ are hydrogen atoms and Z is a carboxylic acid, the compound is either isolated and/or purified.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention also provides compounds and pharmaceutical compositions useful for the treatment of gastrointestinal conditions, having the formula:

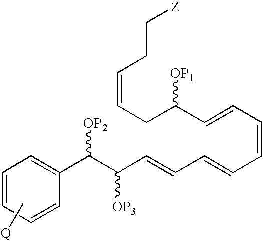

P₁, P₂, P₃, Q and Z are as defined above.

In one particular aspect, P₁, P₂ and P₃ each are hydrogen atoms, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

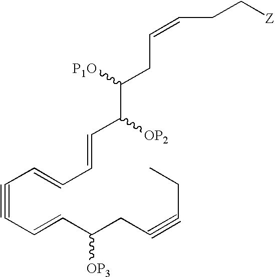

P₁, P₂, P₃ and Z are as defined above.

In one embodiment, P₁, P₂ and P₃ each are hydrogen atoms and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention still further provides compounds and pharmaceutical compositions useful for the treatment of gastrointestinal conditions, having the formula:

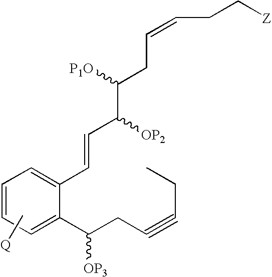

P₁, P₂, P₃, Q and Z are as defined above.

In one particular embodiment, P₁, P₂ and P₃ each are hydrogen atoms, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

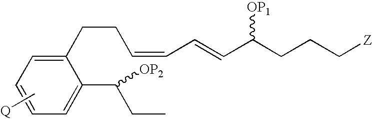

P₁, P₂, Q and Z are as defined above.

In one embodiment, P₁, and P₂ each are hydrogen atoms, each Q is a hydrogen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

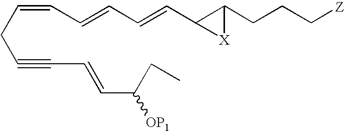

P₁, X and Z are as defined above.

In one aspect, P₁ is a hydrogen atom, X is an oxygen atom and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

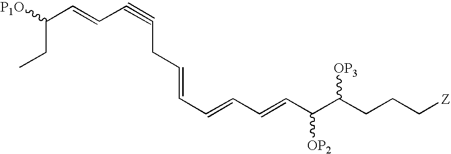

P₁, P₂, P₃ and Z are as defined above.

In one embodiment, P₁, P₂ and P₃ each are hydrogen atoms and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention also provides compounds and pharmaceutical compositions useful for the treatment of gastrointestinal conditions, having the formula:

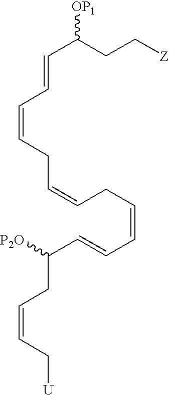

P₁, P₂, U and Z are as defined above.

In one aspect, P₁, and P₂ each are hydrogen atoms, U is a trifluoromethyl group and Z is a carboxylic acid or a carboxylic ester.

In particular, Z is a carboxylic acid, ester, amide, thiocarbamate, carbamate, thioester, thiocarboxamide or a nitrile.

In certain embodiments Z is carboxylic acid, a carboxylic ester, or pharmaceutically acceptable carboxylic acid salt.

The present invention further provides compounds and pharmaceutical compositions useful for the treatment of gastrointestinal conditions, having the formula:

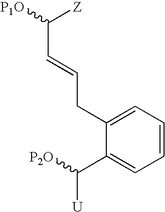

P₁, P₂, U and Z are as defined above. For example, P₁, and P₂ each are hydrogen atoms, U is a trifluoromethyl group and Z is a carboxylic acid or a carboxylic ester.

The present invention, further pertains to dihydroxy-docosahexaenoic acid analogs useful to treat gastrointestinal conditions (diHDHA) having the formula

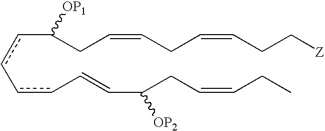

The analogs are designated as 10,17-diHDHAs. P₁ and P₂ are as defined above and can be the same or different. Z is as defined above and in particular can be a carboxylic acid, ester, amide, thiocarbamate, carbamate, thioester, thiocarboxamide or a nitrile. The broken double bond line indicates that either the E or Z isomer is within the scope of the analog(s). In certain aspects, the chiral carbon atom at the 10 position (C-10) has an R configuration. In another aspect, the C-10 carbon atom has an S configuration. In still another aspect, the C-10 carbon atom preferably is as an R/S racemate. Additionally, the chiral carbon atom at the 17 position (C-17) can have an R configuration. Alternatively, the C-17 carbon can preferably have an S configuration. In still yet another aspect, the C-17 carbon can exist as an R/S racemate. In one example, the present invention includes 10,17S-docosatriene, 10,17S-dihydroxy-docosa-4Z,7Z,11E,13,15E,19Z-hexaenoic acid analogs such as 10R/S-OCH₃,17S-HDHA, 10R/S, methoxy-17S hydroxy-docosa-4Z,7Z,11E,13,15E,19Z-hexaenoic acid derivatives.

In certain embodiments, when P₁ and P₂ are hydrogen atoms and Z is a carboxylic acid, the compound is either isolated and/or purified.

In still yet another embodiment, the present invention pertains to diHDHA analogs useful to treat gastrointestinal conditions having the formula

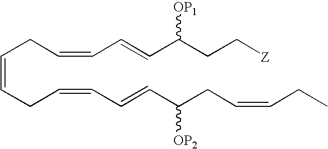

The analogs are designated as 4,17-diHDHAs. P₁, P₂ and Z are as defined above. P₁ and P₂ can be the same or different. In particular, Z can be a carboxylic acid, ester, amide, thiocarbamate, carbamate, thioester, thiocarboxamide or a nitrile. In certain aspects, the chiral carbon atom at the 4 position (C-4) has an R configuration. In another aspect, the C-4 carbon atom preferably has an S configuration. In still another aspect, the C-4 carbon atom is as an R/S racemate. Additionally, the chiral carbon atom at the 17 position (C-17) can have an R configuration. Alternatively, the C-17 carbon can have an S configuration. In still yet another aspect, the C-17 carbon can preferably exist as an R/S racemate.

In certain embodiments, when P₁ and P₂ are hydrogen atoms and Z is a carboxylic acid, the compound is either isolated and/or purified.

For example, the present invention includes 4S,17R/S-diHDHA, 4S,17R/S-dihydroxy-docosa-5E,7Z,10Z,13Z,15E,19Z-hexaenoic acid analogs.

It should be understood that “Z” can be altered from one particular moiety to another by a skilled artisan. In order to accomplish this in some particular instances, one or more groups may require protection. This is also within the skill of an ordinary artisan. For example, a carboxylic ester (Z) can be converted to an amide by treatment with an amine. Such interconversion are known in the art.

In the EPA and DHA analogs, it should be understood that reference to “hydroxyl” stereochemistry is exemplary, and that the term is meant to include protected hydroxyl groups as well as the free hydroxyl group. In certain embodiments, the C-17 position has an R configuration. In other embodiment, the C-17 position has an S configuration. In other aspects, certain embodiments of the invention have an R configuration at the C-18 position.

In certain aspects of the present invention, ASA pathways generate R>S and therefore, 4S,5R/S,7S,8R/S,11R,12R/S16S17R. With respect to species generated from the 15-LO pathway the chirality of C-17 is S, C-16R and C-10, preferably R.

The hydroxyl(s) in the EPA and DHA analogs can be protected by various protecting groups (P), such as those known in the art. An artisan skilled in the art can readily determine which protecting group(s) may be useful for the protection of the hydroxyl group(s). Standard methods are known in the art and are more fully described in literature. For example, suitable protecting groups can be selected by the skilled artisan and are described in Green and Wuts, “Protecting Groups in Organic Synthesis”, John Wiley and Sons, Chapters 5 and 7, 1991, the teachings of which are incorporated herein by reference. Preferred protecting groups include methyl and ethyl ethers, TMS or TIPPS groups, acetate (esters) or propionate groups and glycol ethers, such as ethylene glycol and propylene glycol derivatives.

For example, one or more hydroxyl groups can be treated with a mild base, such as triethylamine in the presence of an acid chloride or silyl chloride to facilitate a reaction between the hydroxyl ion and the halide. Alternatively, an alkyl halide can be reacted with the hydroxyl ion (generated by a base such as lithium diisopropyl amide) to facilitate ether formation.

The compounds can be prepared by methods provided in U.S. patent applications Ser. No. 09/785,866, filed Feb. 16, 2001, entitled “Aspirin Triggered Lipid Mediators” by Charles N. Serhan and Clary B. Clish, Ser. No. 10/639,714, filed Aug. 12, 2003, entitled “Resolvins: Biotemplates for Novel Therapeutic Interventions” by Charles N. Serhan and PCT Applications WO 01/60778, filed Feb. 16, 2001, entitled “Aspirin Triggered Lipid mediators” by Charles N. Serhan and Clary B. Clish and WO 04/014835, filed Aug. 12, 2003, entitled “Resolvins: Biotemplates for Novel Therapeutic Interventions” by Charles N. Serhan, the contents of which are incorporated herein by reference in their entirety.

It should also be understood that for the EPA and DHA analogs, not all hydroxyl groups need be protected. One, two or all three hydroxyl groups can be protected. This can be accomplished by the stoichiometric choice of reagents used to protect the hydroxyl groups. Methods known in the art can be used to separate the di- or tri-protected hydroxy compounds, e.g., HPLC, LC, flash chromatography, gel permeation chromatography, crystallization, distillation, etc.

It should be understood that there are one or more chiral centers in each of the above-identified compounds. It should understood that the present invention encompasses all stereochemical forms, e.g., enantiomers, diastereomers and racemates of each compound. Where asymmetric carbon atoms are present, more than one stereoisomer is possible, and all possible isomeric forms are intended to be included within the structural representations shown. Optically active (R) and (S) isomers may be resolved using conventional techniques known to the ordinarily skilled artisan. The present invention is intended to include the possible diastereiomers as well as the racemic and optically resolved isomers.

The resolvin analogs depicted throughout the specification contain acetylenic and/or ethylenically unsaturated sites. Where carbon carbon double bonds exist, the configurational chemistry can be either cis (E) or trans (Z) and the depictions throughout the specification are not meant to be limiting. The depictions are, in general, presented based upon the configurational chemistry of related DHA or EPA compounds, and although not to be limited by theory, are believed to possess similar configuration chemistry.

Throughout the specification carbon carbon bonds in particular have been “distorted” for ease to show how the bonds may ultimately be positioned relative one to another. For example, it should be understood that acetylenic portions of the resolvins actually do include a geometry of approximately 180 degress, however, for aid in understanding of the synthesis and relationship between the final product(s) and starting materials, such angles have been obfuscated to aid in comprehension.

Throughout the organic synthesis presented below, it should be understood that hydrogenation of acetylenic portions of the resolvin analog may result in one or more products.

It is intended that all possible products are included within this specification. For example, hydrogenation of a diacetylenic resolvin analog can produce up to 8 products (four diene products, i.e., cis, cis; cis, trans; trans, cis; trans, trans) if hydrogenation of both acetylenic portions is completed (this can be monitored by known methods) and four monoacetylene-monoethylene products (cis or trans “monoene”-acetylene; acetylene-cis or trans “monoene”. All products can be separated and identified by HPLC, GC, MS, NMR, IR.

Known techniques in the art can be used to convert the carboxylic acid/ester finctionality of the resolvin analog into carboxamides, thioesters, nitrile, carbamates, thiocarbamates, etc. and are incorporated herein. The appropriate moieties, such as amides, can be further substituted as is known in the art.

In general, the resolvin analogs of the invention are bioactive as alcohols. Enzymatic action or reactive oxygen species attack at the site of inflammation or degradative metabolism. Such interactions with the hydroxyl(s) of the resolvin molecule can eventually reduce physiological activity as depicted below:

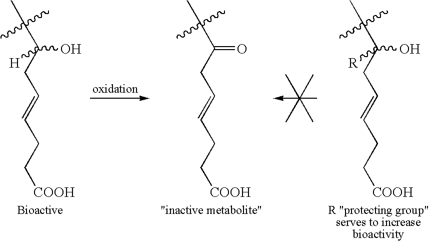

The use of “R” groups with secondary bioactive alcohols, in particular, serves to increase the bioavailability and bioactivity of the resolvin analog by inhibiting or diminishing the potential for oxidation of the alcohol to a ketone producing an inactive metabolite. The R “protecting groups” include, for example, linear and branched, substituted and unsubstituted alkyl groups, aryl groups, alkylaryl groups, phenoxy groups, and halogens.

Generally the use of “R protection chemistry” is not necessary with vicinal diols within the resolvin analog. Typically vicinal diols are not as easily oxidized and therefore, generally do not require such protection by substitution of the hydrogen atom adjacent to the oxygen atom of the hydroxyl group. Although it is generally considered that such protection is not necessary, it is possible to prepare such compounds where each of the vicinal diol hydroxyl groups, independently, could be “protected” by the substitution of the hydrogen atom adjacent to the oxygen atom of the hydroxyl group with an “R protecting group” as described above.

The term “tissue” is intended to include intact cells, blood, blood preparations such as plasma and serum, bones, joints, muscles, smooth muscles, and organs.

The term “subject” is intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species.

When the compounds of the present invention are administered as pharmaceuticals, to humans and mammals, they can be given per se or as a pharmaceutical composition containing, for example, 0.1 to 99.5% (more preferably, 0.5 to 90%) of active ingredient, i.e., at least one EPA or DHA analog, in combination with a pharmaceutically acceptable carrier.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977;66:1-19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention. Such solutions are useful for the treatment of conjunctivitis.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Intravenous injection administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infuslion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 0.1 to about 40 mg per kg per day. For example, between about 0.01 microgram and 20 micrograms, between about 20 micrograms and 100 micrograms and between about 10 micrograms and 200 micrograms of the compounds of the invention are administered per 20 grams of subject weight.

If desired, the effective daily dose of the active compound may be administered as two, three, four, five, six or more sub-doses administered separately at appropriate intervals throughout the day, optionally, in unit dosage forms.

The pharmaceutical compositions of the invention include a “therapeutically effective amount” or a “prophylactically effective amount” of one or more of the EPA or DHA analogs of the invention. A “therapeutically effective amount” refers to an amount effective, at dosages and for periods of time necessary, to achieve the desired therapeutic result, e.g., a diminishment or prevention of effects associated with various disease states or conditions. A therapeutically effective amount of the EPA or DHA analog may vary according to factors such as the disease state, age, sex, and weight of the individual, and the ability of the therapeutic compound to elicit a desired response in the individual. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the EPA or DHA analog and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a EPA or DHA analog of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Delivery of the EPA or DHA analogs of the present invention to the lung by way of inhalation is an important method of treating a variety of respiratory conditions (airway inflammation) noted throughout the specification, including such common local conditions as bronchial asthma and chronic obstructive pulmonary disease. The EPA or DHA analogs can be administered to the lung in the form of an aerosol of particles of respirable size (less than about 10 μm in diameter). The aerosol formulation can be presented as a liquid or a dry powder. In order to assure proper particle size in a liquid aerosol, as a suspension, particles can be prepared in respirable size and then incorporated into the suspension formulation containing a propellant. Alternatively, formulations can be prepared in solution form in order to avoid the concern for proper particle size in the formulation. Solution formulations should be dispensed in a manner that produces particles or droplets of respirable size.

Once prepared an aerosol formulation is filled into an aerosol canister equipped with a metered dose valve. The formulation is dispensed via an actuator adapted to direct the dose from the valve to the subject.

Formulations of the invention can be prepared by combining (i) at least one EPA or DHA analog in an amount sufficient to provide a plurality of therapeutically effective doses; (ii) the water addition in an amount effective to stabilize each of the formulations; (iii) the propellant in an amount sufficient to propel a plurality of doses from an aerosol canister; and (iv) any further optional components e.g. ethanol as a cosolvent; and dispersing the components. The components can be dispersed using a conventional mixer or homogenizer, by shaking, or by ultrasonic energy. Bulk formulation can be transferred to smaller individual aerosol vials by using valve to valve transfer methods, pressure filling or by using conventional cold-fill methods. It is not required that a stabilizer used in a suspension aerosol formulation be soluble in the propellant. Those that are not sufficiently soluble can be coated onto the drug particles in an appropriate amount and the coated particles can then be incorporated in a formulation as described above.

Aerosol canisters equipped with conventional valves, preferably metered dose valves, can be used to deliver the formulations of the invention. Conventional neoprene and buna valve rubbers used in metered dose valves for delivering conventional CFC formulations can be used with formulations containing HFC-134a or HFC-227. Other suitable materials include nitrile rubber such as DB-218 (American Gasket and Rubber, Schiller Park, Ill.) or an EPDM rubber such as Vistalon™ (Exxon), Royalene™ (UniRoyal), bunaEP (Bayer). Also suitable are diaphragms fashioned by extrusion, injection molding or compression molding from a thermoplastic elastomeric material such as FLEXOMER™ GERS 1085 NT polyolefin (Union Carbide).

Formulations of the invention can be contained in conventional aerosol canisters, coated or uncoated, anodized or unanodized, e.g., those of aluminum, glass, stainless steel, polyethylene terephthalate.

The formulation(s) of the invention can be delivered to the respiratory tract and/or lung by oral inhalation in order to effect bronchodilation or in order to treat a condition susceptible of treatment by inhalation, e.g., asthma, chronic obstructive pulmonary disease, etc. as described throughout the specification.

The formulations of the invention can also be delivered by nasal inhalation as known in the art in order to treat or prevent the respiratory conditions mentioned throughout the specification.

While it is possible for a compound of the present invention to be administered alone, it is preferable to administer the compound as a pharmaceutical composition.

The invention features an article of manufacture that contains packaging material and a EPA or DHA analog formulation contained within the packaging material. This formulation contains an at least one EPA or DHA analog and the packaging material contains a label or package insert indicating that the formulation can be administered to the subject to treat one or more conditions as described herein, in an amount, at a frequency, and for a duration effective to treat or prevent such condition(s). Such conditions are mentioned throughout the specification and are incorporated herein by reference. Suitable EPA analogs and DHA analogs are described herein.

More specifically, the invention features an article of manufacture that contains packaging material and at least one EPA or DHA analog contained within the packaging material. The packaging material contains a label or package insert indicating that the formulation can be administered to the subject to asthma in an amount, at a frequency, and for a duration effective treat or prevent symptoms associated with such disease states or conditions discussed throughout this specification.

Materials and Methods

Biogenic Synthesis of 17R-containing HDHA Products:

For scale-up production of diHDHA products, human recombinant cyclooxygenase-2 (COX-2) was expressed in insect Sf9 cells and isolated microsomal fractions were prepared and suspended in Tris Buffer (100 mM,pH 8.0). Aspirin was added (2 mM) (30 min, RT) to assess 17R HDHA formation from DHA (10 uM) before large scale preparations that was confirmed as by LC-MS-MS analysis. Next, DHA (100 mg from Sigma D-2534) was suspended in EtOH and added at 1% v/v to Borate buffer (0.1 mM H₃BO₃, pH 8.5) and vortexed in a round-bottom flask (5 to 10 min under a stream of nitrogen) to form micelle suspensions (optical density >650 nm) to react first with the acetylated-COX-2 (60 min, RT) to generate the 17R oxygenation (see illustration scheme A).

These reaction mixtures were immediately transferred and spun through a Millipore YM-30 centrifuge column for 20 min, RT. Next, isolated potato 5-lipoxygenase purchased from Cayman Chemical was added at 400 ul (in accordance with each preparation's specific enzymatic activity) to 10 ml reactions for 30 min, 4° C. in a round-bottom flask flushed with O₂ rotating in an ice water bath. At time intervals, samples were taken from the reactions to monitor product formation using LC-MS-MS with tandem UV spectra recorded online with a PDA detector with a MeOH:H2O mobile phase and linear gradient.

Next, the 4 position and 7 position hydroperoxy adducts respectively introduced into the 17RHDHA substrate by the actions of the 5-lipoxygenase were reduced as a mixture with the addition of solid grains of sodium borohydride (5 min, RT) and the incubations were stopped with the addition of 2 vol cold MeOH. The diHDHA products were extracted using liquid-liquid acidic ether (pH 3.5) and washed to approximately neutral pH with water. The structures of the 4S,17R and 7S,17R-diHDHA positional isomers were established by LC-MS-MS (using conditions cited in the specification). These compounds were well resolved in rp-HPLC using MeOH:H2O (65:35 v/v) for isolation and biologic actions. 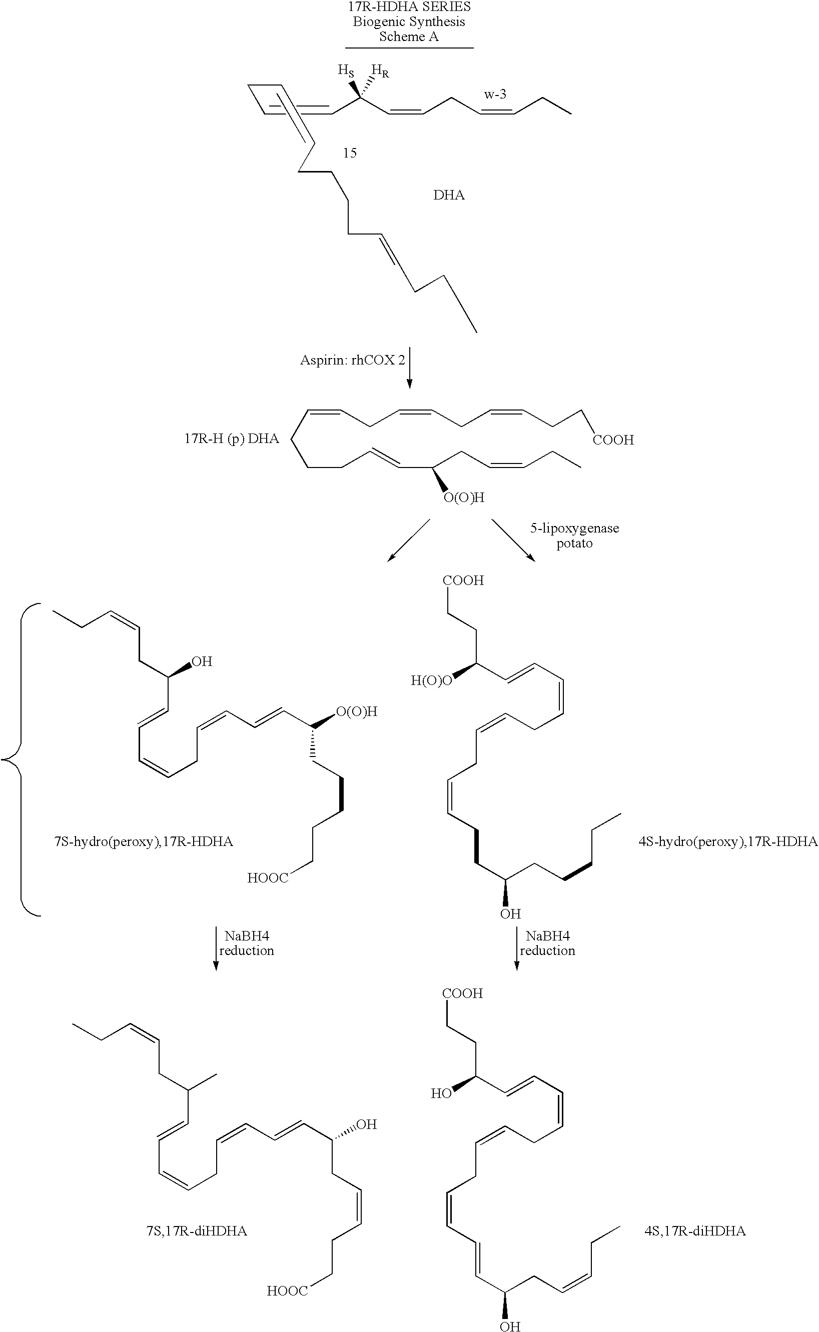
Biogenic Synthesis of 17S containing HDHA Products:

The preparation of the 17S products was carried out using sequential 15-lipoxyenase (soybean lipoxygenase; Sigma) followed by addition of potato 5-lipoxygenase (Cayman Chemical) for scale-up reactions to produce the 4S,17S-diHDHA and 7S,17S-diHDHA shown in scheme B. Both of these lipoxygenases insert molecular oxygen predominantly in the S configuration with antarafacial abstraction of hydrogen at specific positions in DHA (see specification). For these preparations, DHA (100 mg) was suspended in 10 ml Borate buffer (0.1M, pH 9.2), vortexed in a round bottom flask (250 ml vol) to form micelles, and the soybean 15-lipoxygenase was added to the micelle suspension at 4° C. in an ice water bath using spinning rotation for continuous mixing for 30-40 mins to convert DHA to the 17S-H(p)DHA. This hydroperoxy DHA was reduced with the addition of a few grains of NaBH₄ to the flask to produce the corresponding 17S hydroxy-DHA (see Scheme B). Next, the isolated potato 5-lipoxygenase was added to the flask kept at 4° C., pH 9.0 with rotation and oxygen to insert the 4S hydroperoxy- and 7S hydroperoxy- into 17S-HDHA followed by reduction with NaBH4. The reactions were monitored using LC-MSM-MS (vide supra), stopped with 2 vol MeOH, and acidic ether extracted and the positional isomers isolated using RP-HPLC. These 17S-containing products gave similar biologic in murine inflammation and physical properties (i.e. UV chromophores) to their corresponding 17R products, but displayed different retention times in GC-MS. 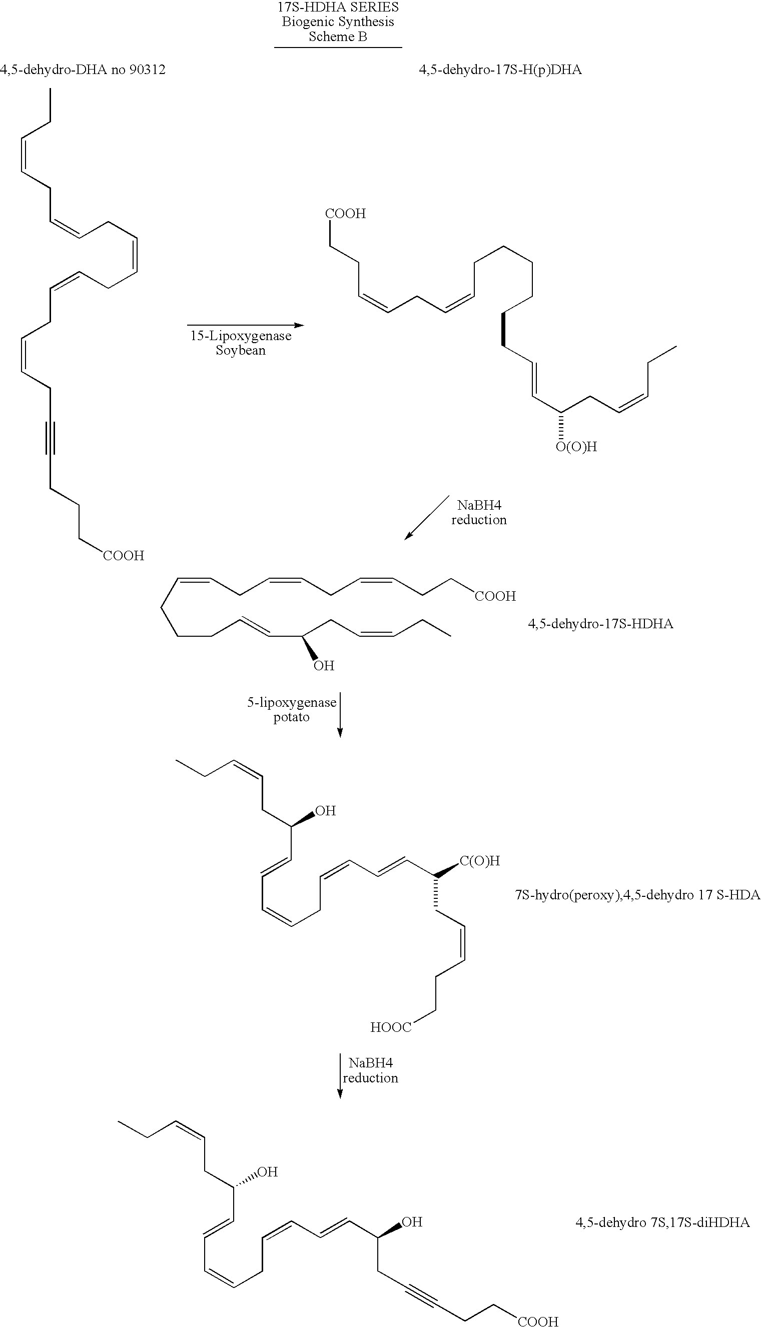
Biogenic Synthesis of 5S,18R/S diHEPA and 5,12,18tri-EPE

(+/−) 18-HEPE was purchased from Cayman Chemical (cat. Number 32840 CAS registry No. 141110-17-0) and used to produce the new EPA-derived compounds (see scheme C) by reactions and procedures described (vide supra) using the potato 5-lipoxygenase for scale up reactions. The racemate 18+/−HEPE (100 ug aliquots in EtOH) was suspended in Borate buffer (pH 9.2) in round-bottom flasks (250 ml) in 0.1% EtOH v/v, vortexed 5-10 min to form micelles and placed at 4° C., rotating as noted above in an ice water bath. The 5-lipoxygenase was added in 25 ul aliquots in two consecutive bolus additions for the isolated enzyme. The first bolus initiated reactions leading to production of 5S,18R/S-diHEPE after NaBH₄ reduction as the main product (see scheme C) generated after 30-40 min reactions as monitored by LC-MS-MS. The second bolus addition of 5-lipoxygenase to the mixture gave rise to the 5,12,18R/S-triHEPA via production of a 5(6)-epoxide intermediate formed by the LTA synthase reaction of the potato 5-lipoxygenase at this pH and substrate concentration. The epoxide opens in an SN2 type reaction in the presence of water adding at the least hindered carbon of the carbonium cation generated at the end (carbon 12) of the conjugated triene system (see below, scheme C). The structures were confirmed by LC-MS-MS and isolated by HPLC for assessment of biologic actions. 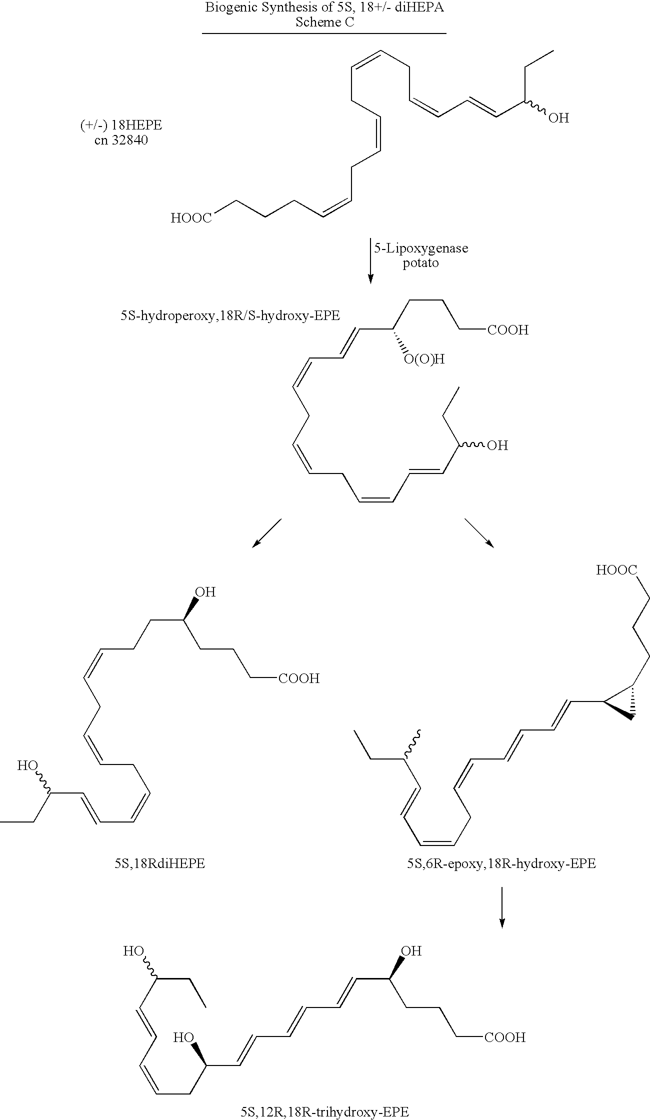
Examples of Analogs Via Biogenic Synthesis:
Synthesis of 4,5-dehydro7S,17S-diHDHA 4,5-dehydro Docosahexaenoic Acid (cat number 90312) was purchased from Cayman Chemical (MI) and used without additional purification to produce analogs for scale-up biological analysis. The 4,5 dehydro DHA in 100 ug aliquot suspensions was prepared in 0.1M Borate Buffer (pH 9.2) in 25 ml round-bottom flask for vortexing and micelle formation before addition of the 15-lipoxygenase in 25 ul aliquots. After reduction with NaBH₄ of the hydroperox-added in the S configuration at position 17, the corresponding alcohol was next converted with the addition of potato 5-lipoxygenase followed by reduction to give the 4,5-dehydro 7S,17S-diHDHA (see scheme D). This scheme can also be used to generate the corresponding 17R containing analogs by substituting the ASA-treat recombinant COX-2 in the position 1 enzyme instead of 15-lipoxygenase.

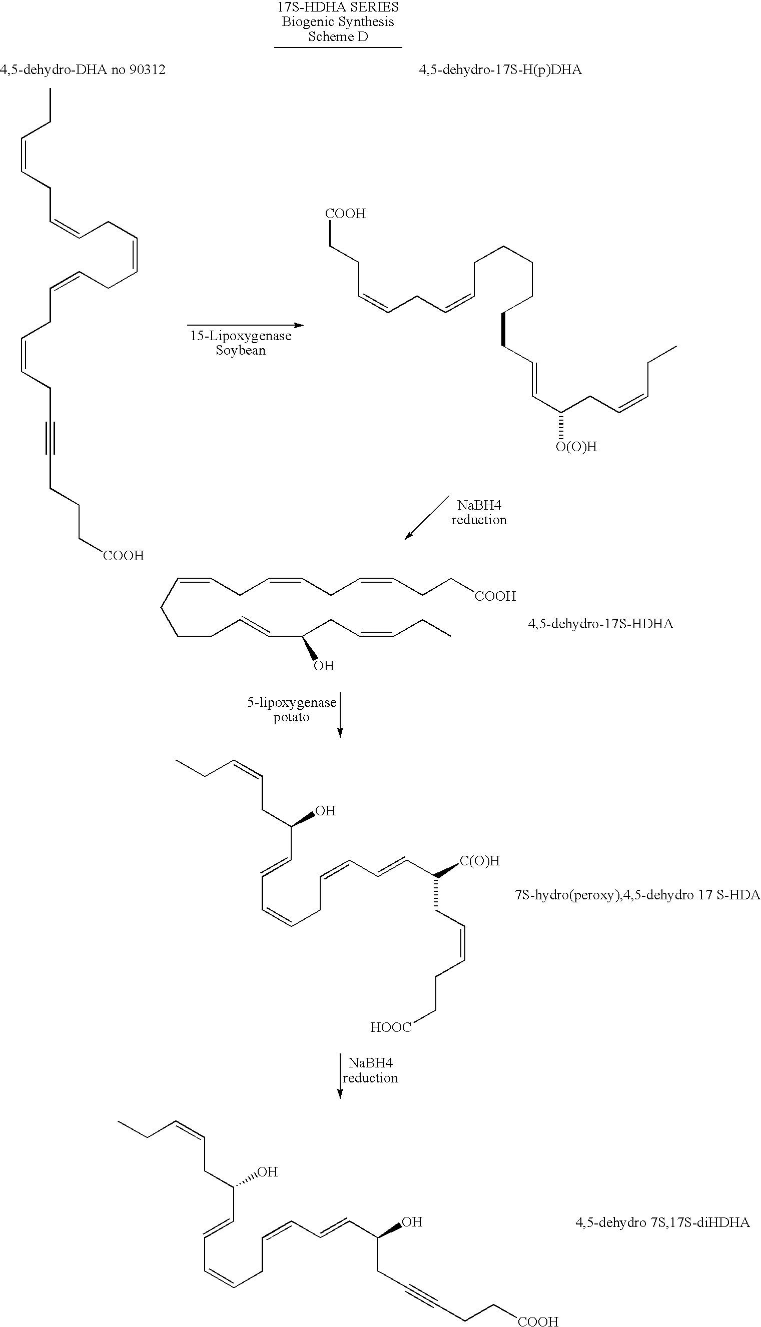

Another example of this route for scale-up is given in Scheme E for the biogenic synthesis of a novel analog, 4,5-dehydro 10,17S-dihydroxy DHA. In short, after addition of 15-LO that was converted to the 17S adduct, a second addition of the soybean 15-LO gave the LTA4-like synthase reaction to yield the 16(17) epoxide of the 4,5dehydro precursor that underwent hydrolysis to give the 4,5-dehydro 10,17S-dihydroxy DHA. This product at a 100 ng dose in murine zymosan A-induced peritonitis gave 40% inhibition of the PMN infiltration, indicating that this Resolvin analog is a potent anti-inflammatory agent in vivo.

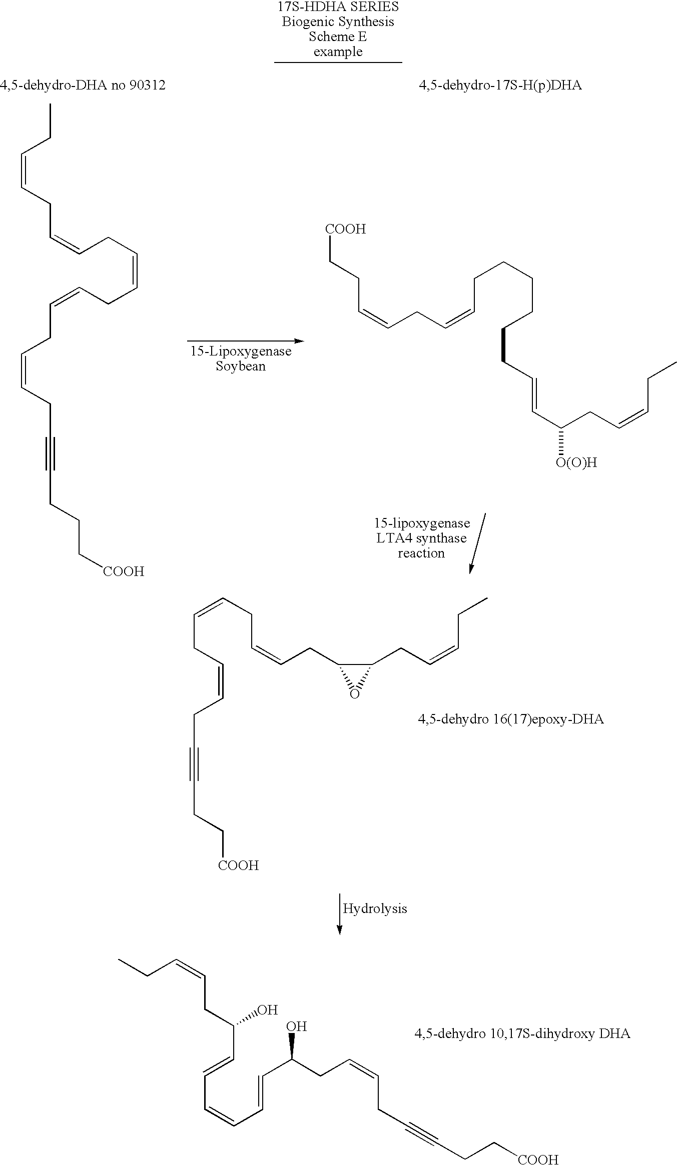

For biogenic synthesis of intermediates and reference compounds, B. megaterium was grown in Bacto Nutrient Broth (Fisher Scientific) at 30° C. with shaking. To prepare standards for 18R-HEPE, a biogenic synthesis was used with B. megaterium sonicates incubated with NADPH (2 mM) and C20:5 (EPA) (330 μM) in 2 M Tris buffer. pH 8.1. Similar conditions were employed to convert LTB₅ (15 μM) to novel products. Incubations were extracted with deuterium-labeled internal standards (15-HETE and C20:4) for LC/MS/MS analysis using a Finnigan LCQ equipped with a LUNA C18-2 (150×2 mm; 5 μM) column and a rapid spectra scanning UV/V is detector. Also, a Chiralcel CB-H column (J. T. Baker) was used to determine R and S alcohol configurations of hydroxy-PUFA using isocratic (hexane/isopropanol 96:4 vol/vol). Detailed procedures for isolation, quantification, and structural determination of lipid-derived mediators were recently reported and used here essentially as described for the elucidation of the novel products.

Mice

6-8-wk-old female BALB/c mice and male FvB mice were obtained from Charles River Laboratories. Mice were provided sterile food and water, kept in microisolator cages, and maintained in the animal facility of Harvard Medical School. All studies were performed under approval of the Harvard Medical School Standing Committee on Animals.

Induction of Peritonitis and Detection of RvE1.

Inflammatory exudates were initiated with intraperitoneal injection of 1 ml zymosan A (1 mg/ml) with 6-8-wk-old male FvB mice. Mice were pre-treated by aspirin (0.5 mg) i.p. followed by EPA (0.3 mg) and zymosan. Peritoneal lavages were collected at 2 h and cells were enumerated. Cell-free exudates were extracted by C18 solid phase extraction with deuterium-LTB₄ (Cascade, UK) as an internal standard for LC-UV-MS/MS analysis using a Finnigan LCQ liquid chromatography ion trap tandem mass spectrometer equipped with a LUNA C18-2 (100×2 mm×5 μm) column and UV diode array detector using mobile phase (methanol:water:acetate at 65:35:0.01) from 0 to 8 min, ramped to methanol 8 to 30 min, with a 0.2 ml/min flow rate.

Induction of TNBS-induced Colitis.

To generate a more chronic T cell-mediated inflammation, BALB/c mice were sensitized with 150 μl of the haptenating agent 2,4,6-trinitrobenzene sulfonic acid (TNBS; Sigma-Aldrich, St. Louis, Mo.) of 2.5% in 50% ethanol by skin painting on day −7. On day 0, 150 μl of 1% TNBS in 50% ethanol was administered intrarectally via a 3.5 F catheter under anesthesia with tribromoethanol. To ensure distribution of the TNBS within the entire colon and cecum, mice were held in a vertical position for 1 min after the instillation. On day 4, mice were killed and immunopathologic characterization was performed as previously described (23).

In Vivo Treatment with RvE1.

RvE1 was prepared by total organic synthesis and qualified by both physical and biological properties (8). RvE1 was administered intraperitoneally (1.0 μg/mouse; 50 μg/kg) on the indicated days before the induction of colitis (prevention mode). Aspirin-triggered lipoxin A₄ analog (ATLa; 15-epi-16-(p-fluoro)phenoxy-LXA₄) was given at the same dose as a direct comparison with RvE1.

Grading of Histologic Change.

The degree of inflammation on microscopic cross sections of the colon was graded semi quantitatively. Severity of colitis was assessed based on five histologic criteria, each graded on a 0-4 scale (0=absent, 1=mild, 2=moderate, 3=severe): mononuclear inflammation, neutrophilic infiltration/crypt abscesses, crypt hyperplasia, mucosal injury/ulceration, and mucosal hypervascularity. The five scores were summed to give a total score. Grading was performed in a blind fashion by the same pathologist.

Myeloperoxidase (MPO) Assay.

Each tissue sample was assessed for PMN content and infiltration. Tissues were homogenized in potassium phosphate buffer (pH 6.0) containing 0.5% hexadecyltrimethylammonium bromide, followed by three cycles of sonication and freeze-thawing. The particulate matter was removed by centrifugation (16,000 g for 20 min), and 75 μl of supernatant was added to 925 μl of potassium phosphate buffer (pH 6.0) containing 0.2 mg/ml o-dianisidine dihydrochloride (Sigma, St. Louis, Mo.) and 0.0006% hydrogen peroxide. Changes in OD were monitored at 460 nm at 25° C., at 30- and 90-s intervals. Calibration curve for conversion of MPO activities to PMN number was performed as in ref. 24.

Detection of TNP-Speciflc Antibodies with ELISA.

Sera were collected on day 4, and anti-trinitrophenyl residue (TNP) antibodies were determined by ELISA as previously described with a minor modification (25). Briefly, 100 mg of OVA was dissolved in 4 ml of 0.05M carbonate-bicarbonate buffer at pH 9.6 followed by adding 1 ml of TNBS 5% solution and incubating for 2 h at room temperature. TNP-OVA was dialyzed in PBS and then stored at −80° C. until use. For the detection of TNP-specific antibodies, TNP-OVA was dissolved at the concentration of 0.1 mg/ml in 0.05M carbonate-bicarbonate buffer at pH 9.6 and 100 μl was placed in each well of 96-well microtiter plates for coating. After blocking with 1% bovine serum albumin in PBS, plates were incubated with appropriately diluted serum. For detection, HRP-conjugated secondary Abs against mouse IgG (Southern Biotech, Birmingham, Ala.) followed by the incubation of TMB Microwell Peroxidase substrate (SureBlue; KPL, Washington, D.C.) as a substrate for HRP were used. One unit, as judged by using the serum of mice that were immunized by 1% TNBS emulsion with Freund's complete adjuvant (Sigma, St. Louis, Mo.) as an internal control, was used.

Quantitative Real-Time PCR.

Total RNA from colon was prepared using Trizol™ reagent (Life Technologies, Rockville, Md.). Real-time PCR was performed using the LightCycler FastStart DNA Master SYBR Green I (Roche Diagnostics GmbH, Mannheim, Germany) according to manufacturer's protocol. Primers for IFN-γ, IL-12p40, TNF-α, IL-4, TGF-β, IL-10, iNOS and glyceraldehyde-3-phosphate dehydrogenase (GAPDH) were designed as described (26). Primers for COX-2 were designed as follows: 5′-AGAAGGAAATGGCTGCAGAA-3′ and 5′-GCTCGGCTTCCAGTATTGAG-3′. Quantities of specific mRNA in the sample were measured according to the corresponding gene-specific standard curve. The relative expression of each gene was normalized against the housekeeping gene GAPDH.

RT-PCR.

Amplification of mouse ChemR23 and GAPDH were carried out with HotStartTaq DNA polymerase (Qiagen, Valencia, Calif.) using specific primers 5′-CTGATCCCCGTCTTCATCAT-3′ and 5′-TGGTGAAGCTCCTGTGACTG-3′, which amplify 376-bp product for ChemR23; 5′-GACCACAGTCCATGCCATCACT-3′ and 5′-TCCACCACCCT GTTGCTGTAG-3′, which amplify 430-bp for GAPDH.

Statistical Analysis.

All results in the figures and text are expressed as mean±SE of n mice per group. Statistical significances were determined by Student's t-test. P values <0.05 were considered as significant difference.

Organic Syntheses of Resolvin Analogs

The following synthetic routes exemplify methods to prepare the resolvin analog families of interest. The preparations are not intended to be limiting but serve as another means to prepare such analogs along more traditional practices and should be considered as complementary to the biogenic syntheses described above. Isolations methods include, column chromatography, HPLC, GC, crystallization and distillation if necessary. Characterization can be accomplished by UV, MS, MS/MS, GC/MS, NMR, etc. One skilled in the art can appreciate the various methods to prepare, isolate and characterize these novel compounds based upon the teachings herein.

The general synthetic schemes provided below depict methods to prepare the various “classes” or families of resolvins encompassed by the present invention. Throughout the syntheses of these families, R groups are used to indicate that various groups can be appended to the resolvin carbon chain. Each R group is independent selected, can be the same or different, and it can be envisioned that each R group is not necessarily present. In those instances, the attachment site would include a hydrogen atom. As described above, the R group is considered a “protecting R group” and can be an substituted or unsubstituted, branched or unbranched alkyl group, arylalkylgroup, alkylaryl group or a halogen atom.

Throughout the synthetic schemes, various hydroxyl protecting groups are depicted. These are not to be considered limiting; these are exemplary protecting groups that can be used and were chosen as illustrative.

The moiety designated as “U” as used throughout the synthetic schemes is described throughout the application and is incorporated herein by reference. “U” as used throughout the synthetic schemes herein is meant to include a terminal carbon atom. The terminal group can be a mono, di or tri substituted methyl group, a methylene (substituted or unsubstituted) attached to a phenoxy group (substituted or unsubstituted), a substituted or unsubstituted aryl group, arylalkyl groups, etc.

“Q” is defined throughout the specification is intended to include one or more substituents positioned about a ring structure. Suitable substituents include, hydrogen atoms, halogen atoms, alkyl groups (substituted and unsubstituted, branched and unbranched), alkylaryl groups, arylalkyl groups, esters, hydroxyls, etc.

The moiety designated as “X” as used throughout the synthetic schemes is described throughout the application and is incorporated herein by reference. “X” as used throughout the synthetic schemes is intended to include, an oxygen atoms, a methylene, a substituted or unsubstituted nitrogen atom or sulfur atom.

As described above, hydrogenation of acetylenic portions of the resolvin can be accomplished to provide one or more products. Selective hydrogenation can provide multiple reaction product dependent upon the degree of hydrogenation that is desired. The resultant product(s) can provide one or more geometric isomers (cis or trans) about the resultant double bond where hydrogenation has taken place. Additionally, selective hydrogenation can provide resolvin analogs that retain one or more acetylenic portions, thus providing still more additional analogs. All analogs are considered part of the present invention and are hereby explicitly incorporated herein. Separation and identification of the compounds can be accomplished by methods known in the art (TLC, HPLC, GC, etc.)

Retention of acetylenic portions within the resolvin analog is considered to be advantageous. The synthesis can be shortened (the hydrogenation step or steps can be eliminated or monitored so that only selective hydrogenation occurs). The resultant acetylenic containing resolvin compounds retain similar bioactivies to the corresponding fully hydrogenated olefinic containing resolving. Additionally, it is believed to be advantageous to avoid hydrogenation of those olefinic bonds that are generated from acetylenic portions which correspond to “cis” configurational isomers with respect to naturally occurring DHA and EPA compounds. That is, retrosynthetically, it is advantageous to prepare DHA and EPA compounds having acetylenic portions where previously cis double bonds existed in the molecule.

For example, Scheme I provides for the general preparation of one class of resolvins

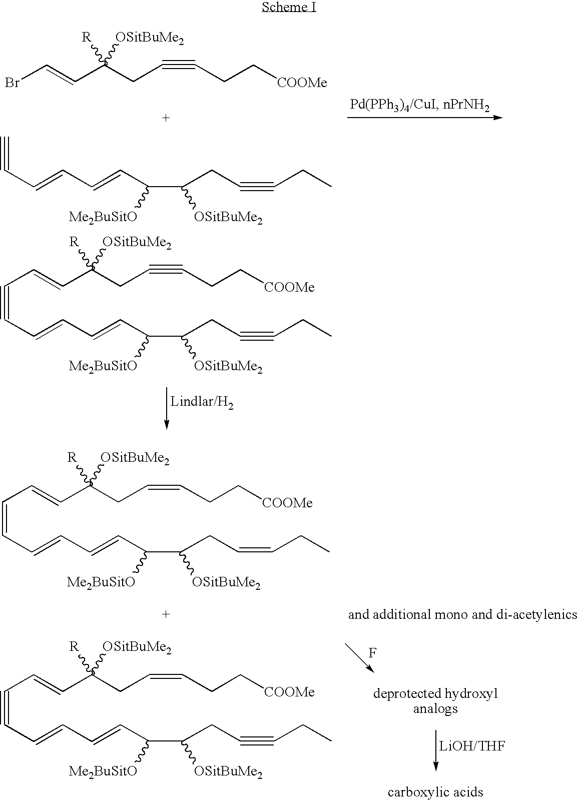

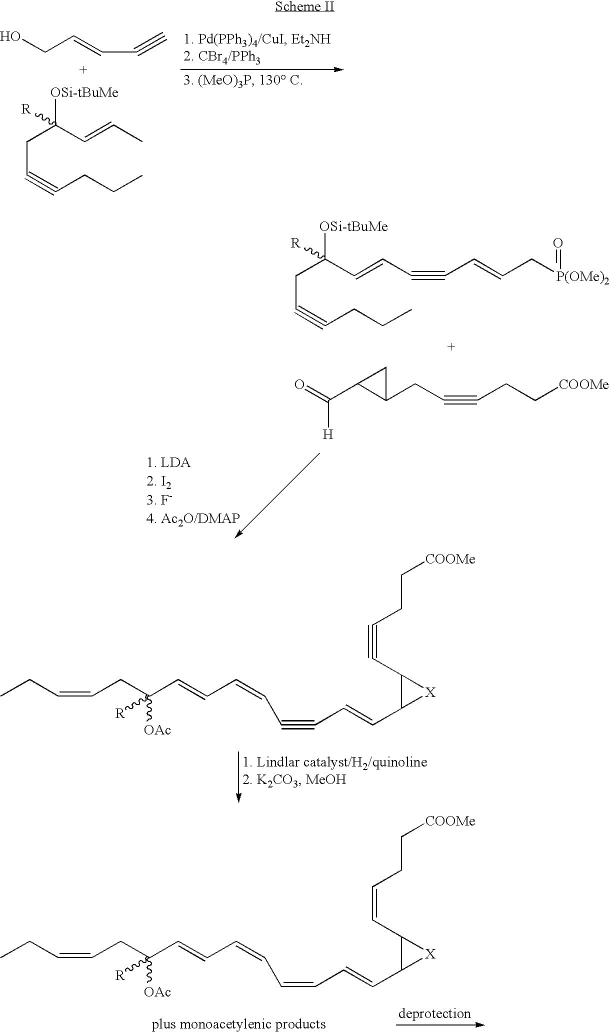

It should be noted, for example, that the “R” groups are used as inhibitors for blocking oxidation of the 7-OH and/or 17-OH, forming ketones at the C-7 or C-17 position(s).

In synthetic scheme II, the synthesis of the 7(8)-methano-analog is based on the biotemplate of the epoxide intermediate in the biosynthesis of bioactive products generated by exudates and cell from DHA as the precursor. In this example, Pd⁰/Cu¹ catalyzed coupling of the vinyl bromide acetylene with the alkynyl alcohol proceeds after bromination and phosphate production to give a phosphonate as an intermediate. The phosphonate is subject to condensation of the lithio derivate with the aldehyde to yield a mixture of A 9-10 cis(E)(Z) trans isomers. These can be converted by treatment with catalytic amounts of iodine. To protect the triple bonds, the silyl protecting group at carbon 17 is replaced by acetate. Lindlar catalytic reduction is used to reduce the triple bonds in quinoline. The product(s) are deacetylated to give the stable cyclopropyl 7(8) methano analog of the equivalent labile epoxide analog that is involved in resolvin biosynthesis of exudates in vivo and/or in cells, such as microlial cells of the brain or human leukocytes.

These analogs are important therapeutics because in addition to acting at the site/receptor for 7,8,17R-triHDHA resolvin as a mimetic to stop leukocyte recruitment as an agonist to stimulate/promote resolution and to pharmacologically inhibit inflammation, it also serves as an inhibitor of enzymes in vivo. Thus the compounds inhibit proinflammatory lipid mediators such as leukotrienes and also lead to an accumulation in situ of upstream resolvins in the biosynthetic pathway. Thus, these class of compounds serve a dual purpose: mimetic of resolvin 7, 8,17-tri-HDHA and as a substrate level inhibitor.

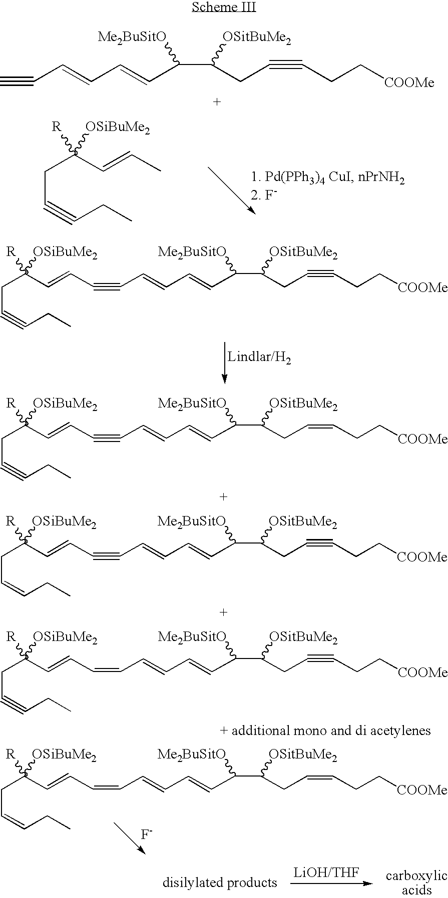

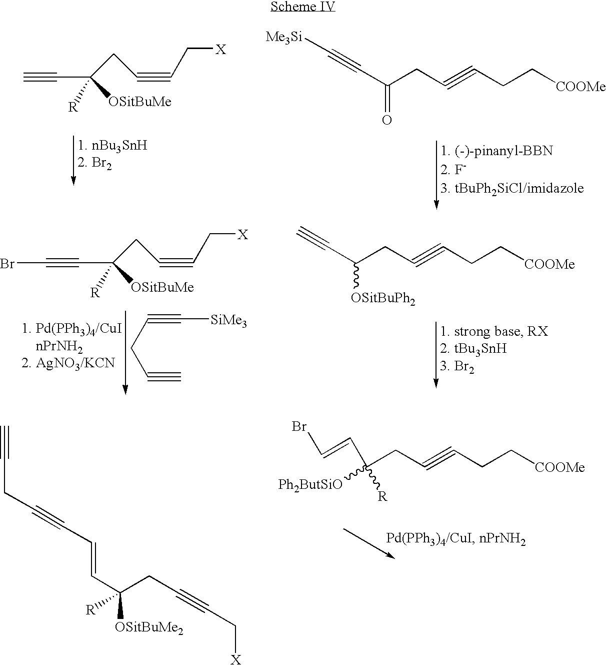

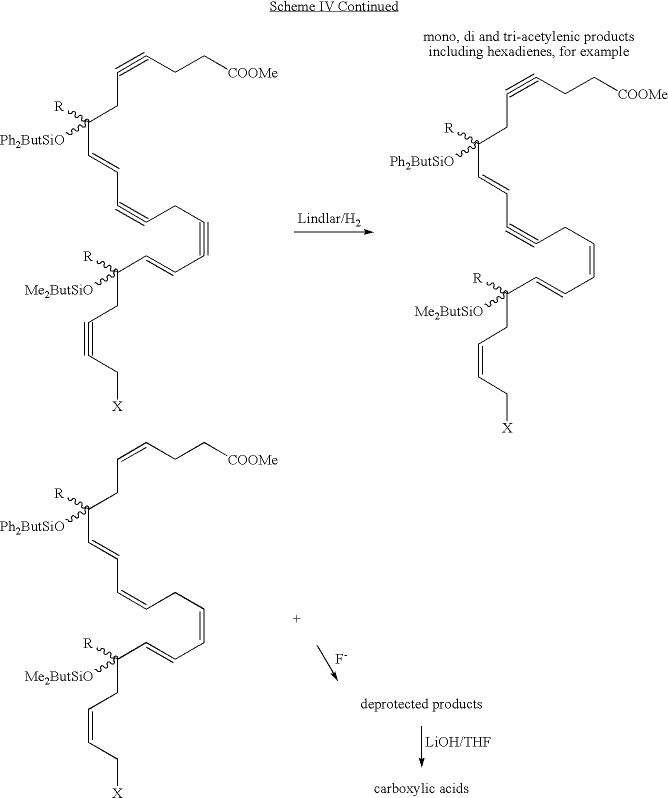

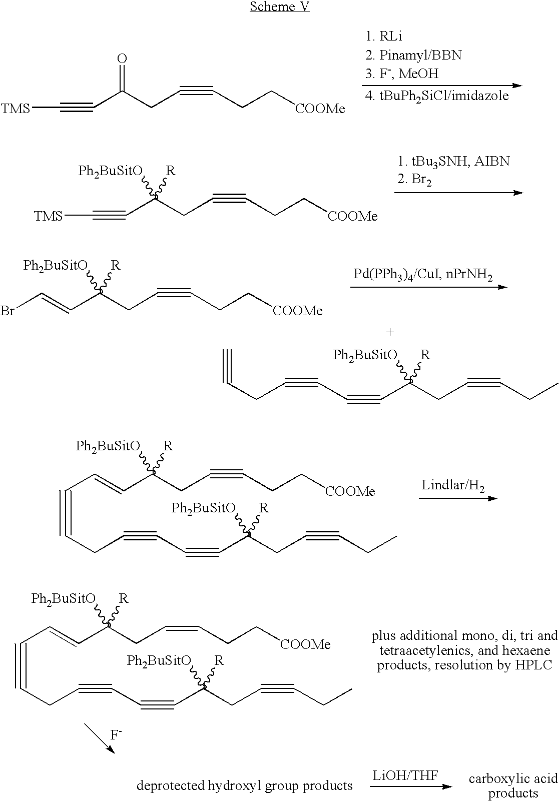

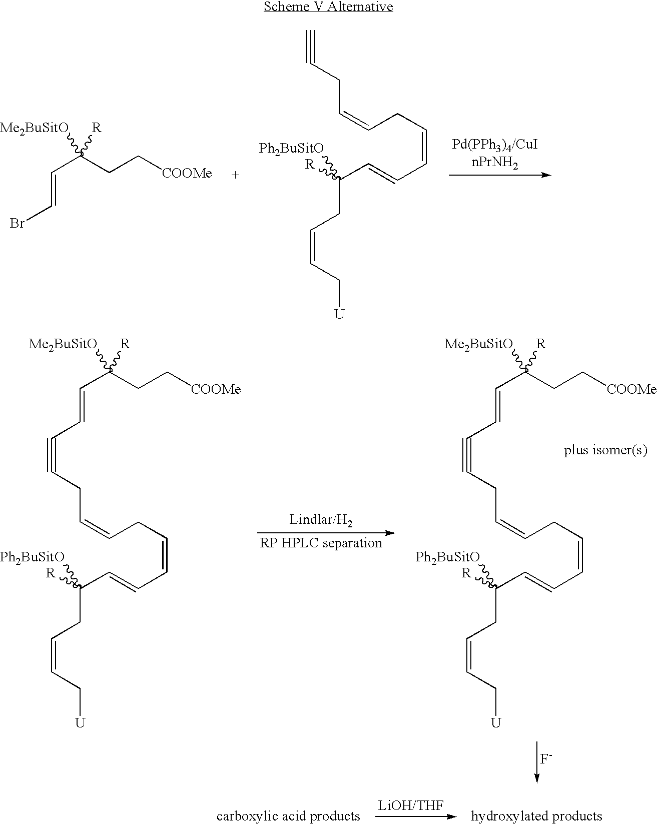

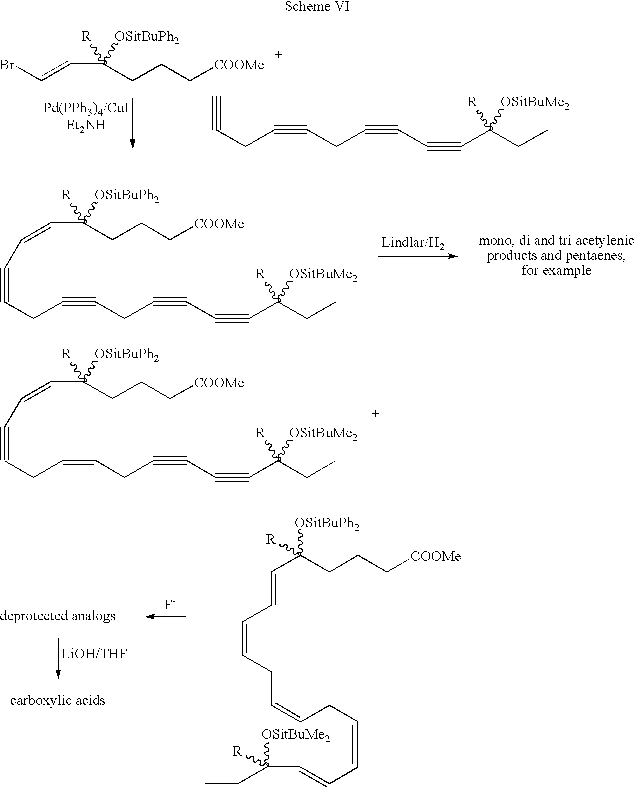

Scheme VI represents another class of compounds, where again, “protection” of the potentially oxidizable 5 and/or 18 hydroxyls to ketones. Use of “R” groups, as described herein, provides the ability to prevent the oxidation, and therefore the bioavailability of the bioactive compound.

The analogs within synthetic scheme VI can be prepared by coupling the vinyl bromide as prepared in K. C. Nicolaou et al. Angew. Chem. Int. Ed. Engl 30 (1991) 1100-1116) and coupled using Pd/Cu coupling chemistry. The resultant intermediate can be selectively hydrogenated with the Lindlar catalyst and hydrogen to produce various acetylenic products, as well as penatene containing products. Deprotection of the alcohols and conversion to carboxylic acids, esters, etc. can be accomplished by known methods.

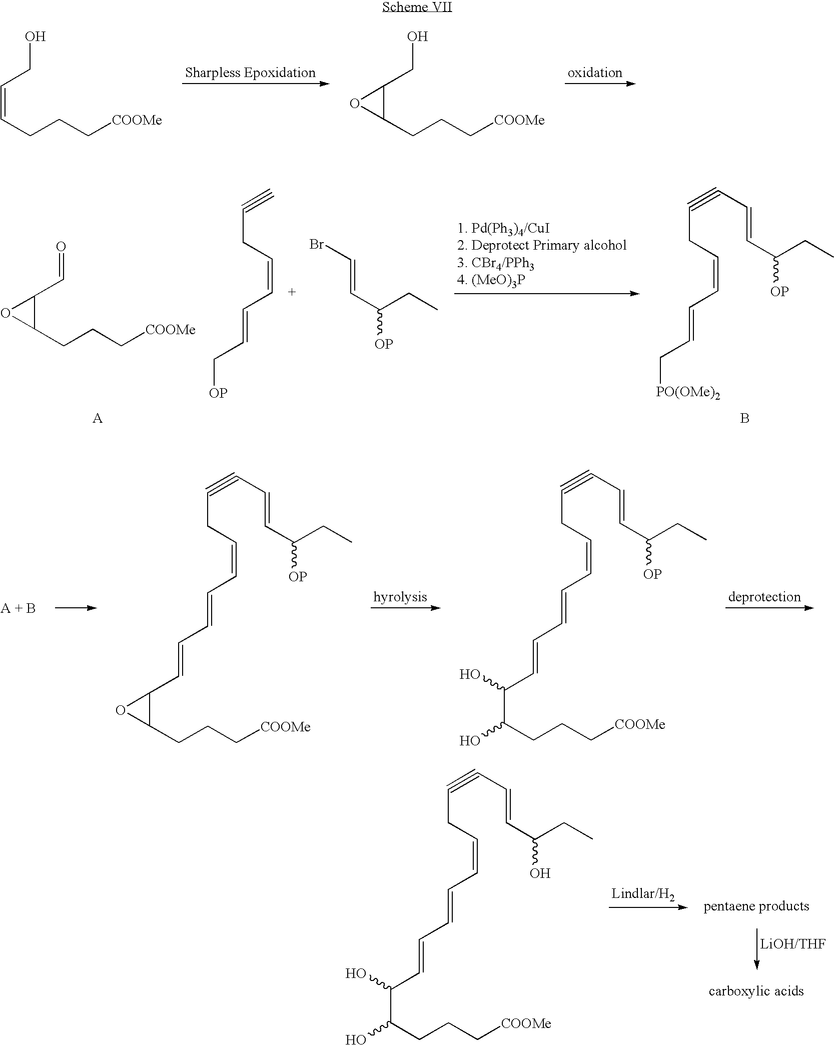

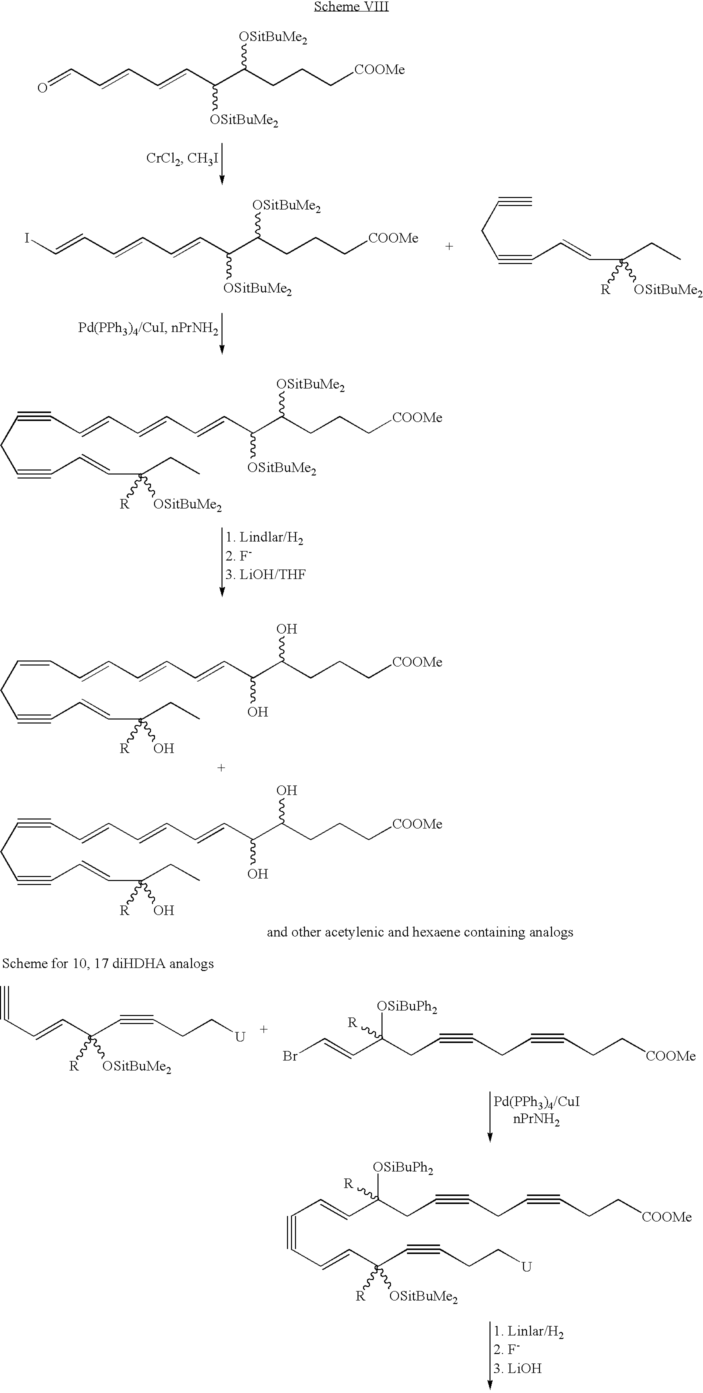

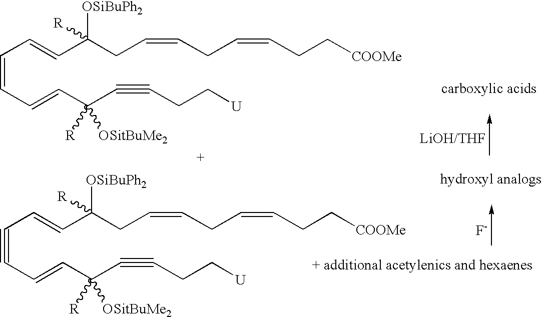

10,17 diHDHA's produced via 15-lipoxygenase action on DHA (pH of about 8.5) under conditions that favor hydroperoxidation at the 17 position of DHA which is then converted into the 16,17 epoxide. The 16(17) epoxide carries the conjugated triene chromophore and opens via a carbonium cation intermediate with OH attack at the 10 position to afford 10,17-diHDHA. Human tissues and isolated cells produce this via the 15-lipoxygenase as well as additional enzymes. This compound has been prepared by using soybean 15-lipoxygenase with DHA as the substrate at a pH of about 8.5, presented in micelle configuration. The 10,17 di-HDHA was isolated using RP-HPLC as described herein. It was found that the 10,17 di-HDHA inhibited both PMN migration into the peritoneum (zymosan induced peritonitis) of mice given Zymosan and inflammation. Hence, protection at the C-10 hyroxyl position with an “R protecting group” should prevent metabolic conversion and increase stability and activation to block PMN infiltration and acute inflammation.

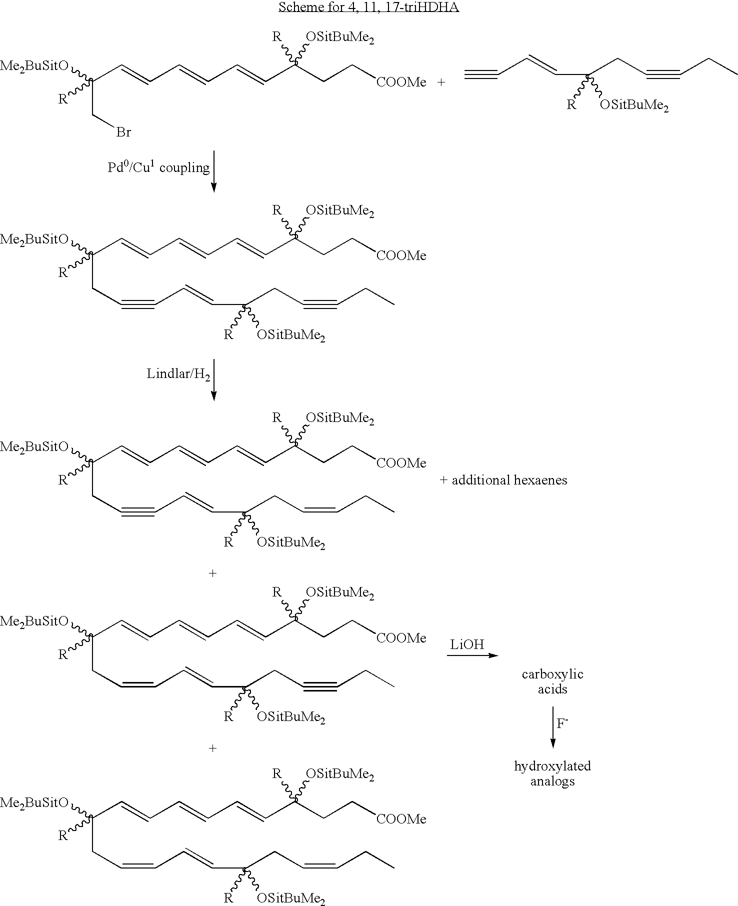

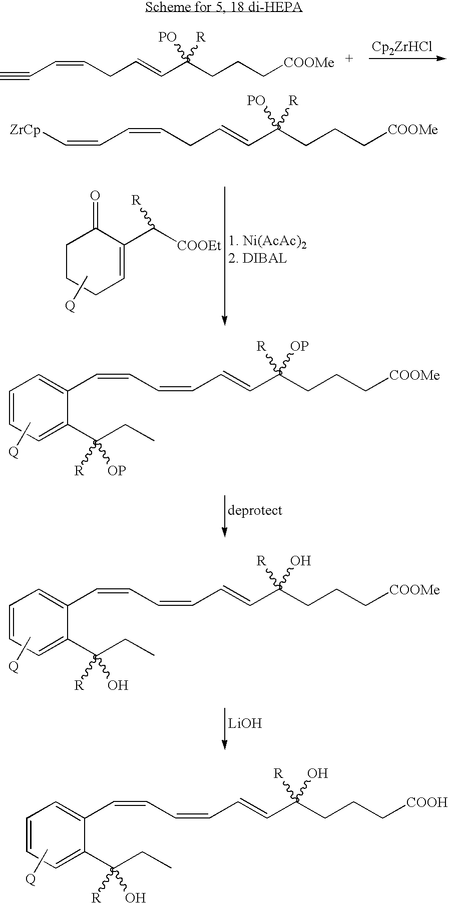

The preparation of 5,18-diHEPA analogs is achieved using a conjugated addition of a vinyl zirconium reagent 3-(1-octen-1-yl)cyclohexanone as in Sun, R. C., M. Okabe, D. L. Coffen, and J. Schwartz. 1992. Conjugate addition of a vinylzirconium reagent: 3-(1-octen-1-yl)cyclopentanone (cyclopentanone, 3-(1-octenyl)-, (E)-). In Organic Syntheses, vol. 71. L. E. Overman, editor. Organic Syntheses, Inc., Notre Dame, Ind. 83-88 using Schwartz's reagent as prepared in Buchwald, S. L., S. J. LaMaire, R. B. Nielsen, B. T. Watson, and S. M. King. 1992. Schwartz's reagent (zirconium, chlorobis(h5-2,4-cyclopentadien-1-yl)hydro-). In Organic Synthesis, vol. 71. L. E. Overman, editor. Organic Syntheses, Inc., Notre Dame, Ind. to constructthe zirconiated intermediate. Treatment with DIBAL as in Ishiyama, T., N. Miyaura, and A. Suzuki. 1992. Palladium(0)-catalyzed reaction of 9-alkyl-9-borabicyclo[3.3.1]nonane with 1-bromo-1-phenylthioethene: 4-(3-cyclohexenyl)-2-phenylthio-1-butene. In Organic Syntheses, vol. 71. L. E. Overman, editor. Organic Syntheses, Inc., Notre Dame, Ind. provides the di-HEPA ring containing analog. It should be understood that the cyclohexanone reagent can be substituted with any number of substituents, thereby providing the resultant substituted or unsubstituted aromatic ring within the di-HEPA analog.

Again, it should be noted that inclusion of an “R protecting group” at C-5 and/or C-18 positions helps to inhibit oxidation of the hydroxyl group to a ketone. Additionally, it is believed that the use of a ring within the structure helps to constrain confirmation about the molecule and affects receptor ligand interaction(s). 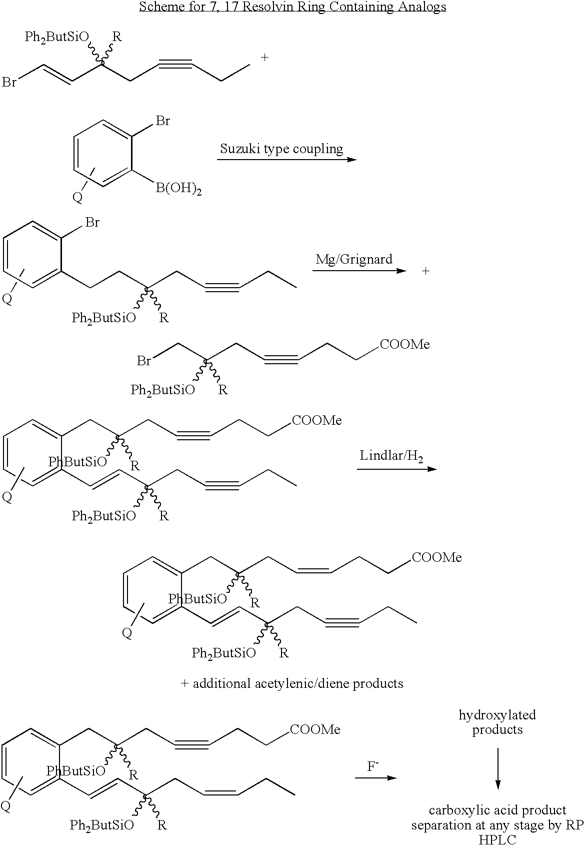 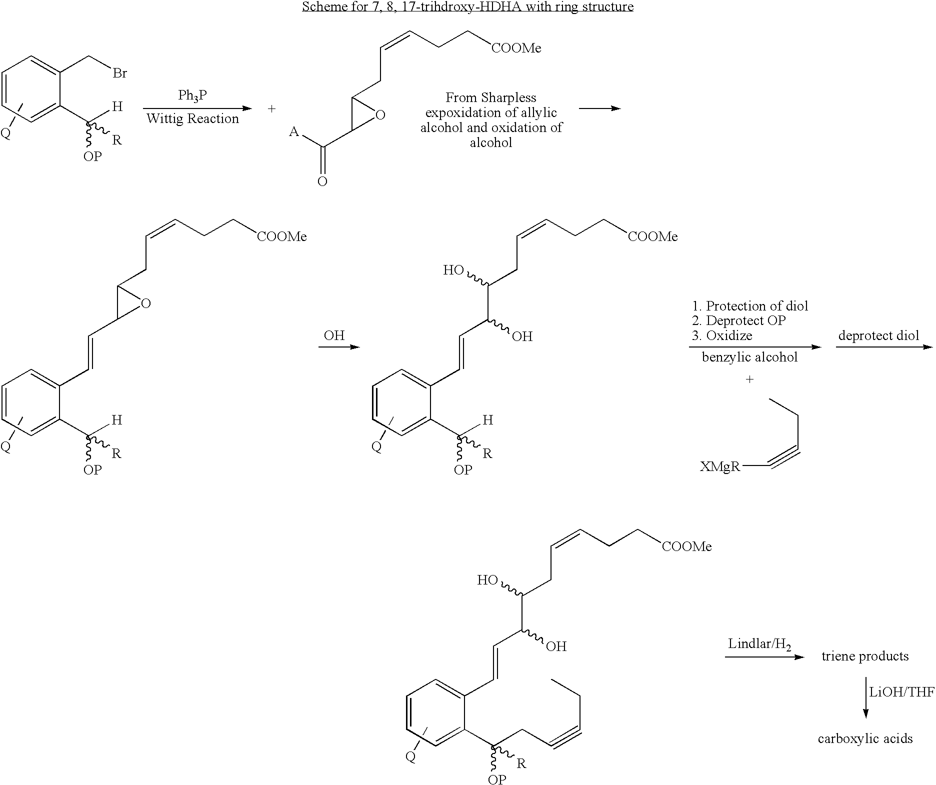 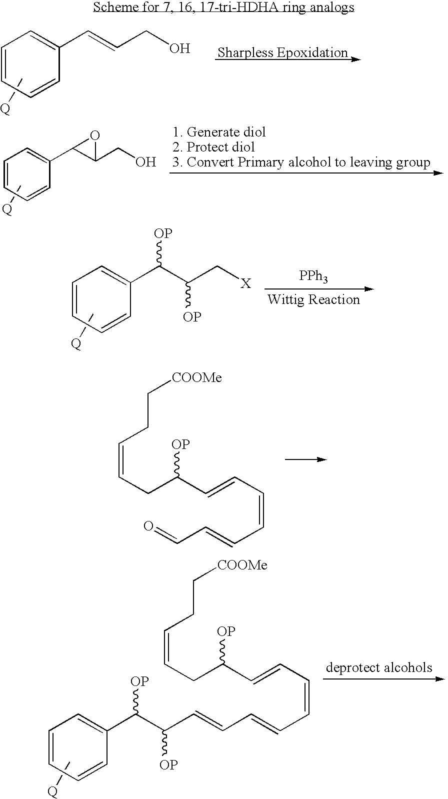
Results
RvE1 Formation in Peritoneal Inflammatory Exudates

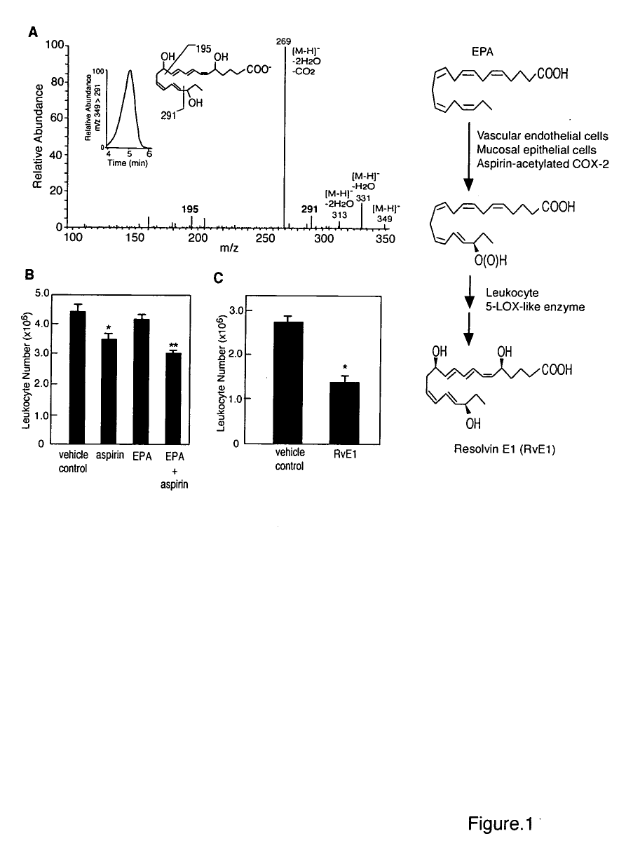

During the course of an acute inflammatory challenge associated with administration of zymosan intraperitoneally, formation of RvE1 was documented in the inflammatory exudates using a LC-UV-MS/MS mediator-lipidomic analysis after injection of EPA and aspirin (FIG. 1A). Selected ion chromatograms and ions present within the MS/MS were consistent with the production of RvE1 with a parent ion at m/z 349=[M-H]⁻ and characteristic product ions at m/z 291 and 195 that are denoted in the inset. These findings are consistent with previous work (7) indicating that murine inflammatory exudates exposed in vivo to EPA and aspirin produced the trihydroxy-containing product RvE1 presumably via the activities of leukocyte 5-lipoxygenase (LO) and aspirin-acetylated cyclooxygenase (COX)-2. Peritoneal inflammatory cells, predominantly PMNs, were 30% lower in EPA- and aspirin-treated mice when endogenous RvE1 was present in the peritoneal exudates (FIG. 1B). Moreover, exogenous administration of as little as 100 ng/mouse of RvE1 reduced leukocyte infiltration by 50% (FIG. 1C). In contrast, reduction product 18R-HEPE from the metabolic precursor 18R-H(p)EPE was much less potent. RvE1 proved to be more potent than indomethacin, a well-characterized non-steroidal anti-inflammatory drug (11), which reduced leukocyte numbers in the exudate by only approximately 20% in this model.

RvE1 Protects Mice from TNBS Colitis

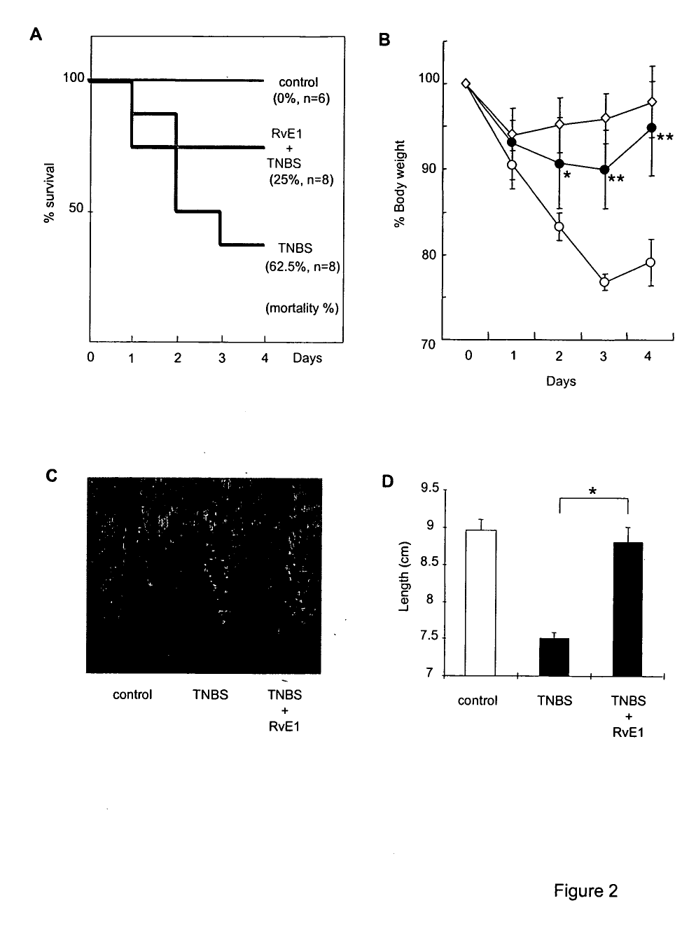

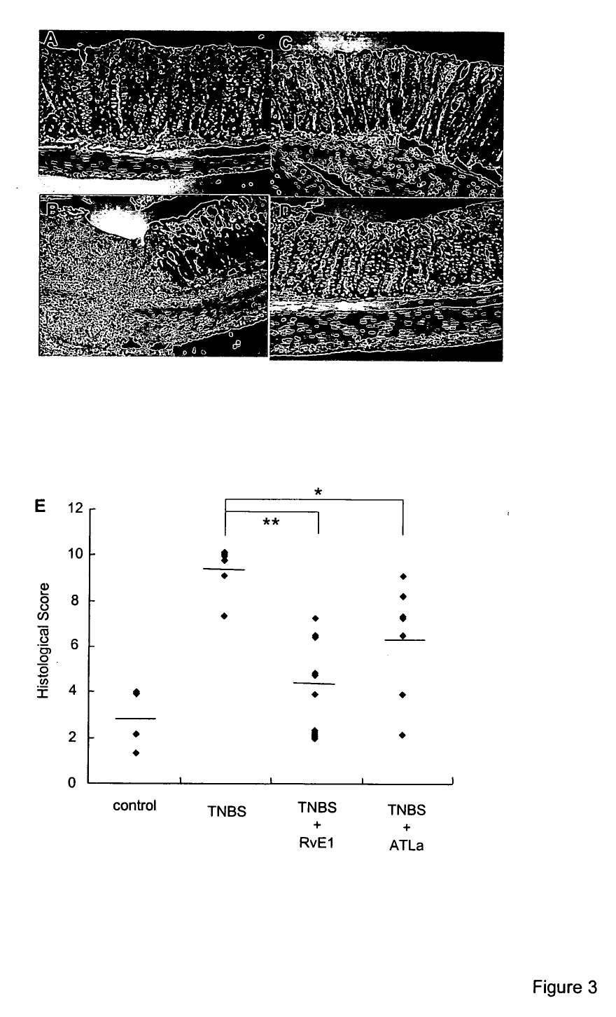

Next the effects of RvE1 were assessed in the TNBS colitis model. After sensitization to TNBS by skin painting, male BALB/c mice (6-8 wk old) were subjected to intrarectal administration of TNBS (1.5 mg/mouse in 50% ethanol). Severe illness that was characterized by bloody diarrhea and severe wasting disease was observed. The treatment of mice with RvE1 (1.0 μg/mouse; 0.05 mg/kg) reduced overall mortality, which was 25% and 62.5%, respectively (RvE1 treatment in comparison to the TNBS groups alone) (FIG. 2A). Animals that received TNBS in association with control vehicle experienced severe weight loss (FIG. 2B, open circle). In contrast, mice that received RvE1 experienced less weight loss (FIG. 2B, closed circle). This is directly reflected in the levels of macroscopic injury observed in that mice treated with RvE1 in that they did not exhibit significant shortening and thickening of the colon (FIG. 2C and 2D). Consistent with these macroscopic changes, mice treated with control vehicle exhibited marked transmural infiltration with inflammatory cells such as PMNs, monocytes, and lymphocytes and injury with ulceration (FIG. 3B). In contrast, mice treated with RvE1 exhibited less severe histologic features of colitis (FIG. 3C). For purpose of direct comparison, aspirin-triggered lipoxin A4 stable analog (ATLa), another aspirin-triggered lipid mediator which proved to be protective against dextran sodium sulfate (DSS)-induced colitis (12), also provided dramatic protection in the TNBS-colitis model (FIG. 3D). When quantified by a histologic scoring system for evidence of inflammation and injury, these histologic differences were highly significant (FIG. 3E).

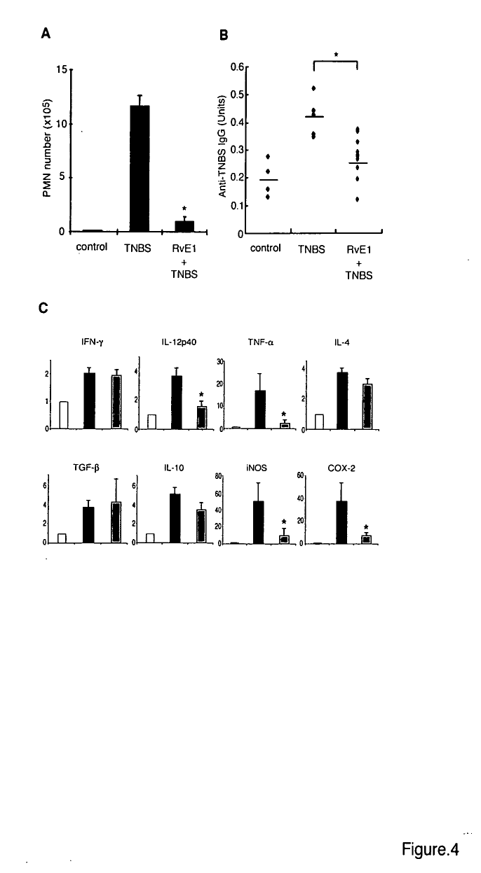

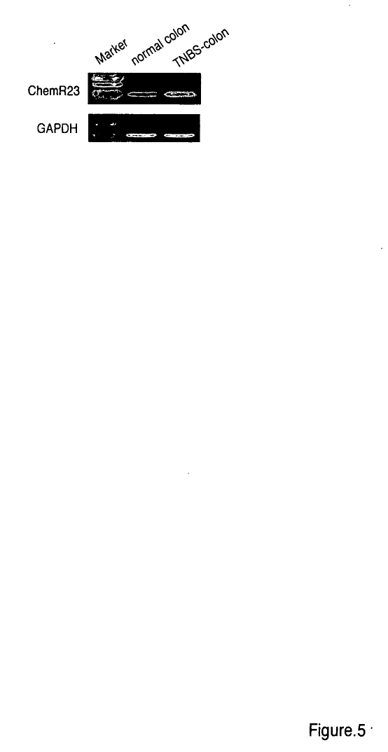

In addition to the histological scores, mice treated with RvE1 exhibited significantly lower levels of myeloperoxidase (MPO) activity when compared to mice treated with the control vehicle, suggesting reduced leukocyte infiltration in colon tissues (FIG. 4A). Serum anti-TNBS IgG level was also decreased by RvE1 treatment, suggesting attenuation of antigen presentation and B-cell production of IgG (FIG. 4B); a measure of the level of adaptive immunity. To determine whether this RvE1-mediated protection from colitis was associated with alterations in pro-inflammatory gene expression, mRNA levels in colon were determined by RT-PCR (FIG. 4C). This study revealed a significant reduction in TNF-α, IL-12p40, iNOS and COX-2 from mice that received RvE1. Interestingly, direct effects were not observed for the T-cell cytokines such as IFN-γ and IL-4 and IL-10. Also TGF-β did not show significant differences. Recently, an RvE1 receptor was identified in human and mouse as a G-protein coupled receptor ChemR23 (8). Murine ChemR23 mRNA was expressed in mouse colon, and was slightly increased in levels in colons obtained from TNBS-treated mice (FIG. 5).

It was demonstrated that resolvins, such as RvE1, exhibit potent anti-inflammatory activity, are generated in the course of inflammation in vivo from EPA when aspirin is administrated. RvE1 reduced leukocyte infiltration, turned off pro-inflammatory gene expression and prevented the development of severe experimental colitis in mice. Together, these observations provide a therapeutic method of resolving intestinal inflammation in vivo.

The concept that novel endogenous counterregulatory pathways of anti-inflammation occur in vivo through generation of resolvins are of interest given the potency of RvE1 observed herein. Hence, understanding the regulation of these natural endogenous anti-inflammatory products is important in order to optimize the potential utility of this novel pathway in vivo. During acute inflammation, inflammatory cells expressing COX-2 treated with aspirin transform EPA via insertion of molecular oxygen in the R configuration to yield 18R-H(p)EPE (7). Acetylation of COX-2 with aspirin treatment promotes the generation of 18R-HEPE which may also account for some of the bioactivity profile of aspirin. Without aspirin, 18R-HEPE could also be generated by bacterial cytochrome P450 monooxygenase (7,13). Once formed, 18R-HEPE is then further converted via cell-cell interactions and the sequential action of leukocyte lipoxygenase reaction that leads to formation of 5S,12R,18R-trihydroxy-6Z,8E,10E,14Z,16E-eicosapentaenoic acid (RvE1) (8).

In Crohn's disease, neutrophil recruitment to the intestinal wall and an excessive activation of macrophages and T helper 1 (Th1) cells leads to the enhanced production of pro-inflammatory cytokines such as TNF-α. This cytokine milieu favors an amplification of the inflammatory cascade of additional inflammatory mediators, destructive enzymes, and free radicals that cause tissue damage (9,10). The relapsing and remitting course of inflammatory bowel disease (IBD), together with the spontaneous resolution, imply the existence of an endogenous resolution signal. In addition to resolvins, it is now appreciated that several new families of endogenous anti-inflammatory and/or pro-resolution mediators are generated during a host response including TGF-β, IL-10, PPARγ agonists and lipoxins (14-17). Studies in humans suggested that omega-3 PUFA such as EPA and DHA are protective in reducing the rates of relapse in Crohn's disease (4,5). These results raise the intriguing possibility that omega-3 PUFA is a precursor of novel endogenous resolution signals. Indeed, the present results demonstrate the endogenous conversion of EPA and formation of the novel Resolvin mediator RvE1 in inflamed loci. Moreover, evidence is provided that RvE1 when administered exogenously protects against the development of TNBS-colitis by reducing local inflammation.

Hapten-induced colitis such as TNBS colitis is a useful model to study the early or initiating events in the development of mucosal inflammation (18). In this model intestinal inflammation develops as a result of the covalent binding of the haptenizing agent to autologous host proteins with subsequent stimulation of a delayed-type hypersensitivity to TNBS-modified self antigens in the context of an exaggerated innate immune response generating IL-12 (19). Although the relationship of this model to human disease is not entirely identical, this hapten-induced colitis model displays Crohn's Disease-like features, most notably transmural leukocytic inflammation and predominant Th1 activity of mucosal leukocytes. RvE1 treatment dramatically reduced leukocyte infiltration, pro-inflammatory gene expression (i.e. IL-12p40, TNF-α) and anti-TNBS IgG level, suggesting the immunoregulatory action of RvE1 in both innate and acquired limbs of the mucosal immune response. It is of note that 15-epi-lipoxin A4, another aspirin-triggered lipid mediator, is also protective against gastric damage induced by dextran sodium sulfate or aspirin (12). Along these lines, during the course of the present manuscript submission, the 3-oxo-ATL stable analog was shown to be protective against TNBS colitis (20). RvE1 and ATL have different receptors, namely ChemR23 and ALX, which share structural similarity but different tissue distributions (8). Both receptors are expressed in colon (FIG. 5 and cf. ref. 20). Hence these aspirin-triggered lipid mediators, namely RvE1 and ATL, act at different sites to protect colon; whether these lipid mediators are additive or synergistic in their actions in colon protection remains for further studies.

Results from both human and animal studies indicate that TNF-α plays a critical role in pathogenesis of IBD. For example, the overexpression of TNF-α in mice results in the development of a Crohn's Disease-like phenotype and TNF-α deficient mice or anti-TNF-α treatment ameliorates intestinal inflammation in several animal models (21). More importantly, blockade of TNF-α is now a widely employed therapeutic strategy in the management of Crohn's disease and rheumatoid arthritis (22). TNF-α directly induces NF-κB mediated up-regulation of adhesion molecules and inflammatory mediators and participates in apoptosis. The dramatic decrease in TNF-α and iNOS expression by as little as 0.05 mg/kg of RvE1 and complete protection against the development of TNBS colitis argue strongly that formation as well as administration of RvE1 and related compounds can offer a new avenue for protection of mucosal inflammation and injury.

Taken together, the present invention provides for a novel endogenous anti-inflammatory lipid mediator RvE1 whose activity is the basis for some of the beneficial actions of omega-3 EPA in human diseases.

### REFERENCES

1. Weiss, U. eds. (2002) Nature 420: 845-891
2. GISSI-prevenzione investigators. (1999) Lancet 354: 447-455
3. Freedman, S. D., Blanco, P. G., Zaman, M. M., Shea, J. C., Ollero, M., Hopper, I. K., Weed, D. A., Gelrud, A., Regan, M. M., Lapostata, M., Alvarez, J. G., and O'Sullivan, B. P. (2004) N. Engl. J. Med. 350: 560-569
4. Belluzzi, A., Brignola, C., Campieri, M., Pera, A., Boschi, S., and Miglioli, M. (1996) N. Engl. J. Med. 334: 1557-1560
5. Teitelbaum, J. E., Walker, W. A. (2001) J. Nutr. Biochem. 12: 21-32
6. Serhan, C. N., Gotlinger, K., Hong, S., and Arita, M. (2004) Prostaglandins Other Lipid Mediat. 73:155-72
7. Serhan, C. N., Clish, C. B., Brannon, J., Colgan, S. P., Chiang, N., and Gronert, K. (2000) J. Exp. Med. 192:1197-1204
8. Arita, M., Bianchini, F., Aliberti, J., Sher, A., Chiang, N., Hong, S., Yang, R., Petasis, N., Serhan, C. N. (2005) J. Exp. Med. 201: 713-722
9. Blumberg, R. S., Strober, W. (2001) JAMA 285: 643-647
10. Podolsky, D. K. (2002) N. Engl. J. Med. 347: 417-429.
11. Wallace, J. L., Chapman, K., McKnight, W. (1999) Br. J. Pharmacol. 126: 1200-1204
12. Gewirtz, A. T., Collier-Hyams, L. S., Young, A. N., Kuncharzik, T., Guilford, W. J., Parkinson J. F., Williams, I. R., Neish, A. S., Madara, J. L. (2002) J. Immunol. 168: 5260-5267
13. Capdevila, J. H., Wei, S., Helvig, C., Falck, J. R., Belosludtsev, Y., Trunan, G., Graham-Lorence, S. E., Peterson, J. A. (1996) J. Biol. Chem. 271: 22663-22671
14. Nathan, C. (2002) Nature 420: 846-852
15. Serhan, C. N. (2002) Curr. Med. Chem. 1: 177-192
16. Lawrence, T., Willoughby, D. A., Gilroy, D. W. (2002) Nat. Rev. Immunol. 2: 787-795
17. Gilroy, D. W., Lawrence, T., Perretti, M., Rossi, G. (2004) Nat. Rev. Drug Disc. 3: 401-18. Strober, W., Fuss, I. J., and Blumberg, R. S. (2002) Annu. Rev. Immunol. 20: 495-549
19. Bouma, G., Kaushiva, A., Strober, W. (2002) Gastroenterology 123: 554-565.
20. Fiorucci, S., Wallace, J. L., Mencarelli, A., Distrutti, E., Rizzo, G., Farneti, S., Morelli, A., Tseng, J., Suramanyam, B., Guilford, W. J., Parkinson, J. F. (2004) Proc. Natl. Acad. Sci. USA. 101: 15736-15741
21. Neurath, M. F., Fuss, I., Pasparakis, M., Alexopoulou, L., Haralambous, S., Meyer zum Buschenfelde, K. H., Strober, W., Kollias, G. (1997) Eur. J. immunol. 27: 1743-1750.
22. Blam, M. E., Stein, R. B., Lichteinstein, G. R. (2001) Am. J. Gastroenterol. 96: 1977-1997.
23. Nieuwenhuis, E. E., Neurath, M. F., Corazza, N., lijima, H., Trgovcich, J., Wirtz, S., Glickman, J., Bailey, D., Yoshida, M., Galle, P. R., Kronenberg, M., Birkenbach, M., Blumberg, R. S. (2002) Proc. Natl. Acad. Sci. USA. 99:16951-16956
24. Devchand, P. R., Arita, M., Hong, S., Bannenberg, G., Moussignac, R. Gronert, K., Serhan, C. N. (2003) FASEB J. 17: 652-659
25. Dohi, T., Fujihashi, K., Kiyono, H., E1son, C. O., McGhee, J. R. (2000) Gastroenterology. 119: 724-733
26. Overbergh, L., Valckx, D., Waer, M., Mathieu, C. (1999) Cytokine. 11: 305-312

Although the present invention has been described with reference to preferred embodiments, persons skilled in the art will recognize that changes may be made in form and detail without departing from the spirit and scope of the invention. All publications and references cited herein, including those in the background section, are expressly incorporated herein by reference in their entirety.

I claim:

1. A method to treat or prevent a gastrointestinal condition, comprising administering to a subject a compound selected from: 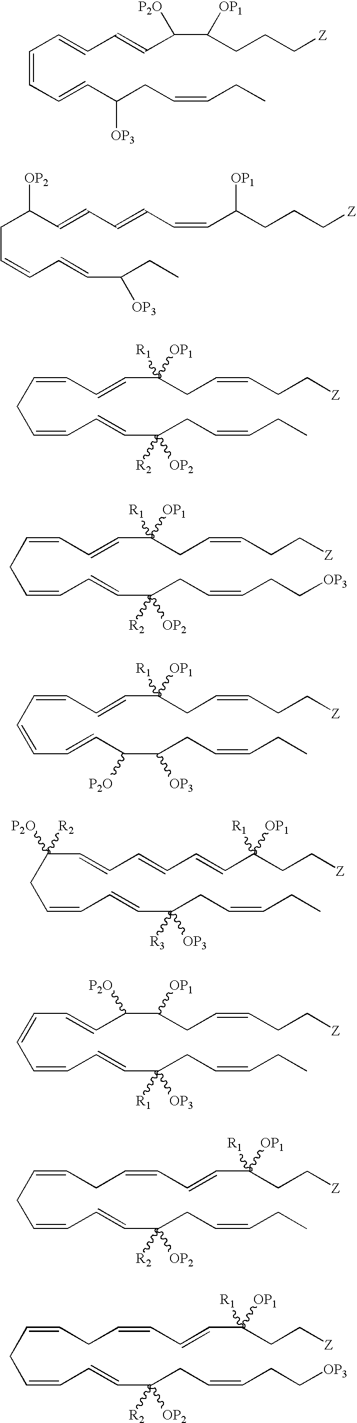 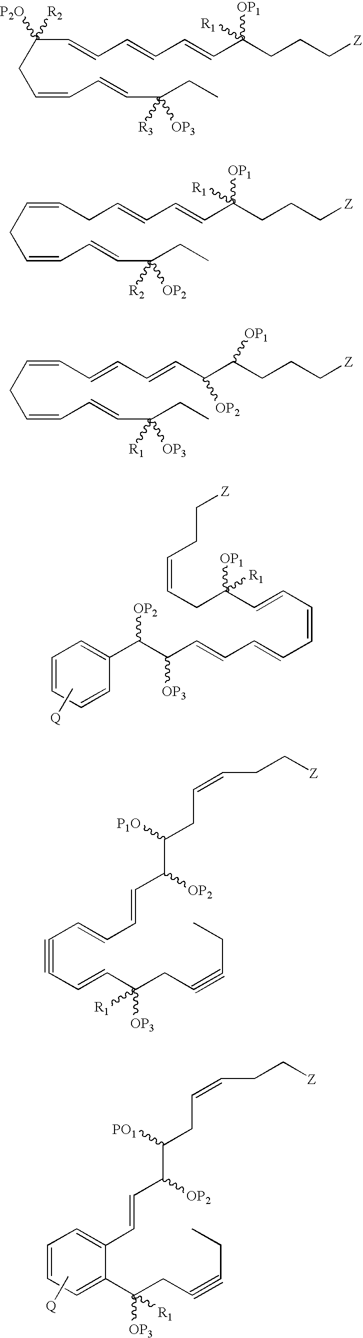 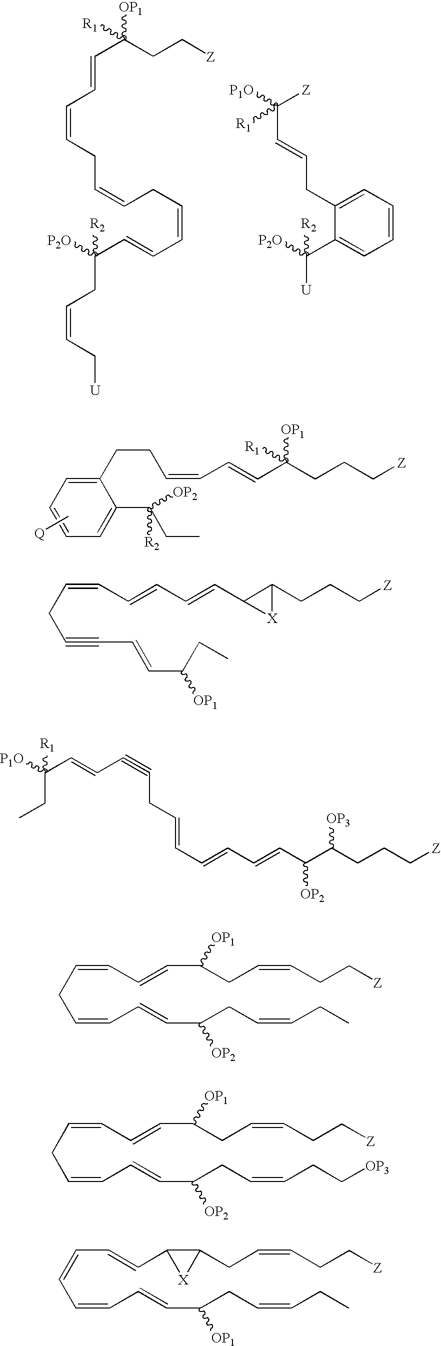 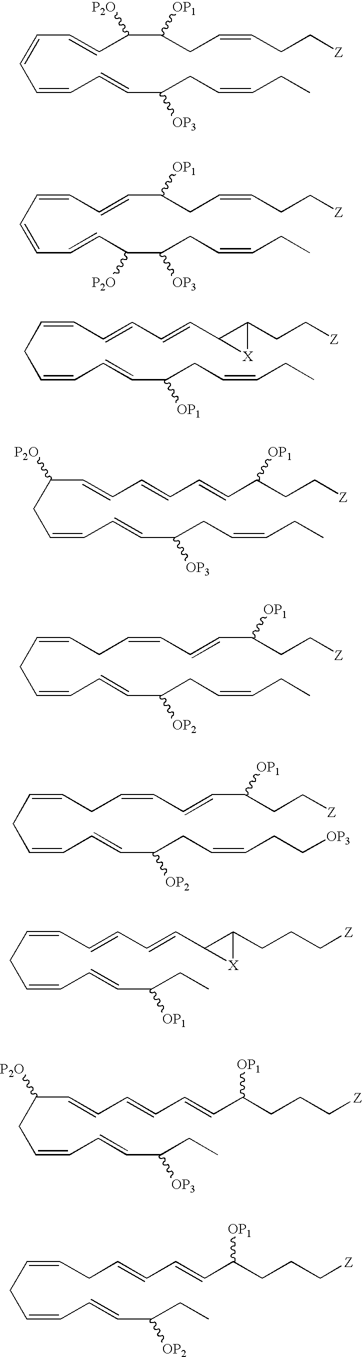 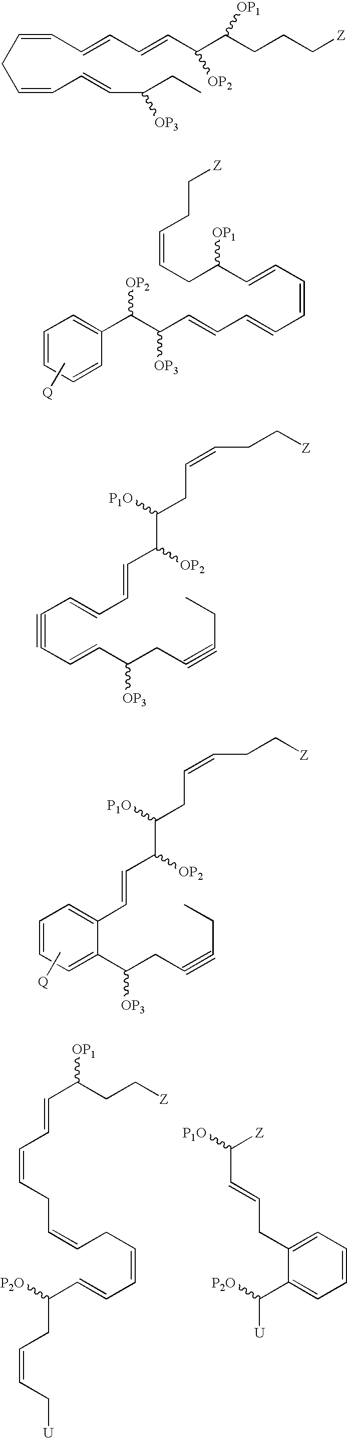 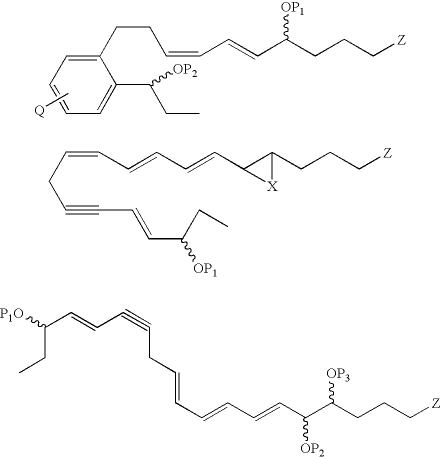
wherein P₁, P₂ and P₃, if present, each individually are protecting groups, hydrogen atoms or combinations thereof;
wherein R₁, R₂ and R₃, if present, each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof;
wherein Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;
each R^{a}, if present, is independently selected from the group consisting of hydrogen, (C1-C6)alkyl, (C3-C8)cycloalkyl, cyclohexyl, (C4-C11)cycloalkylalkyl, (C5-C10)aryl, phenyl, (C6-C16)arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;
each R^{b}, if present, is a suitable group independently selected from the group consisting of ═O, —OR^{d}, (C1-C3)haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]NR^{c}R^{c}, —[NHC(NH)]ₙNR^{c}R^{c} and —[NR^{a}C(NR^{a}) NR^{c}R^{c};
each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;
each n, independently, if present, is an integer from 0 to 3;
each R^{d}, independently, if present, is a protecting group or R^{a};
wherein X, if present, is a substituted or unsubstituted methylene, an oxygen atom, a substituted or unsubstituted nitrogen atom, or a sulfur atom;
wherein Q, if present, represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group;
wherein U, if present, is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;
wherein the double bond configurations of the compounds can be either cis or trans;
and pharmaceutically acceptable salts thereof.

2. The method of claim 1, wherein Z is a carboxylic acid, ester, a pharmaceutically acceptable carboxylic acid salt, or prodrug thereof.

3. The method of claim 1, wherein the C-5, C-6, C-12, C-13, C-14, C-15, C-16, C-17, C-18, C-19 or C-20, each individually, if present, has an R configuration if it is a chiral center.

4. The method of claim 1, wherein the C-5, C-6, C-12, C-13, C-14, C-15, C-16, C-17, C-18, C-19 or C-20, each individually, if present, has an S configuration if it is a chiral center.

5. The method of claim 1, wherein the C-5, C-6, C-12, C-13, C-14, C-15, C-16, C-17, C-18, C-19 or C-20, each individually, if present, has an R/S configuration if it is a chiral center.

6. The method of claim 1, wherein the gastrointestinal condition is ulcerative colitis, Crohn's disease, infectious enteritis, antibiotic associative diarrhea, clostridium difficile colitis, microscopic or lymphocytic colitis, collagenous colitis, colon polyps, familial polyps, familial polyposis syndrome, Gardner's Syndrome, helicobacter pylori, irritable bowel syndrome, nonspecific diarrheal illnesses, and intestinal cancers.

7. The method of claim 1, wherein the gastrointestinal condition is gastrointestinal inflammation.

8. A method to treat or prevent a gastrointestinal condition, comprising administering to a subject a pharmaceutical composition comprising compound selected from: 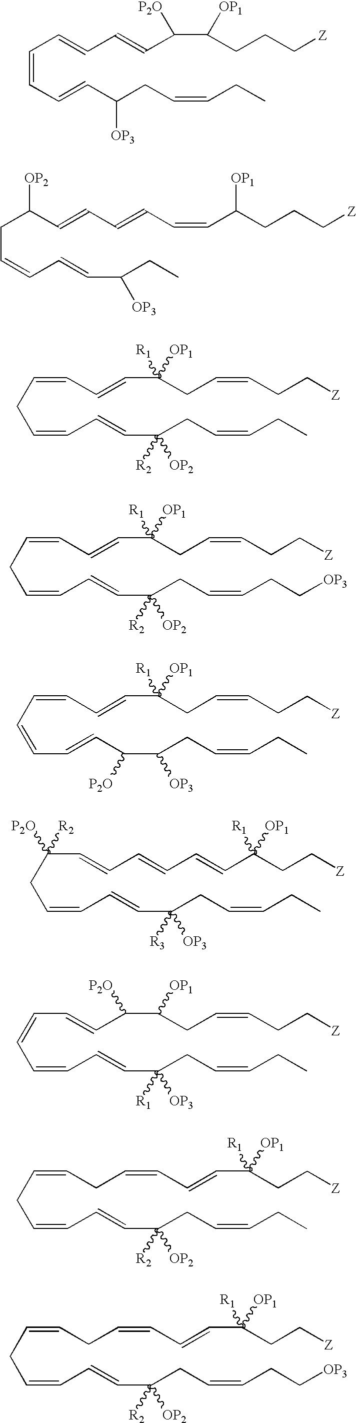 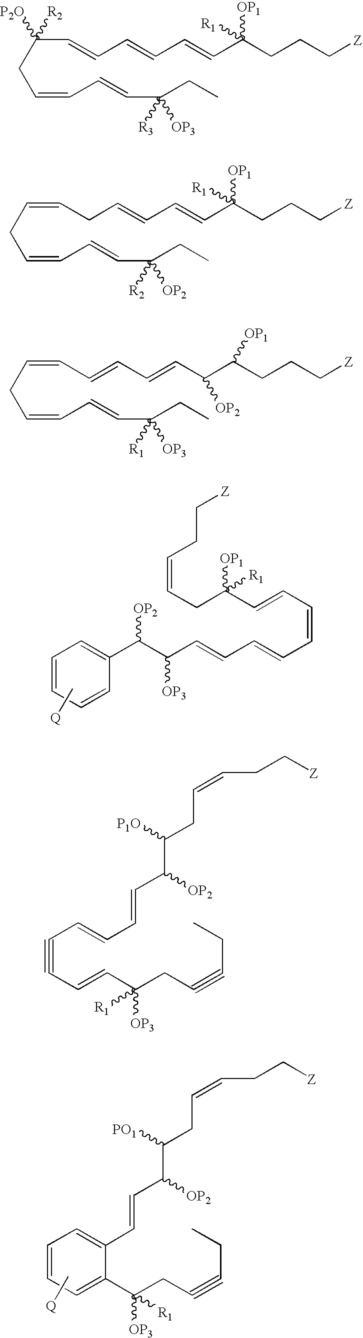 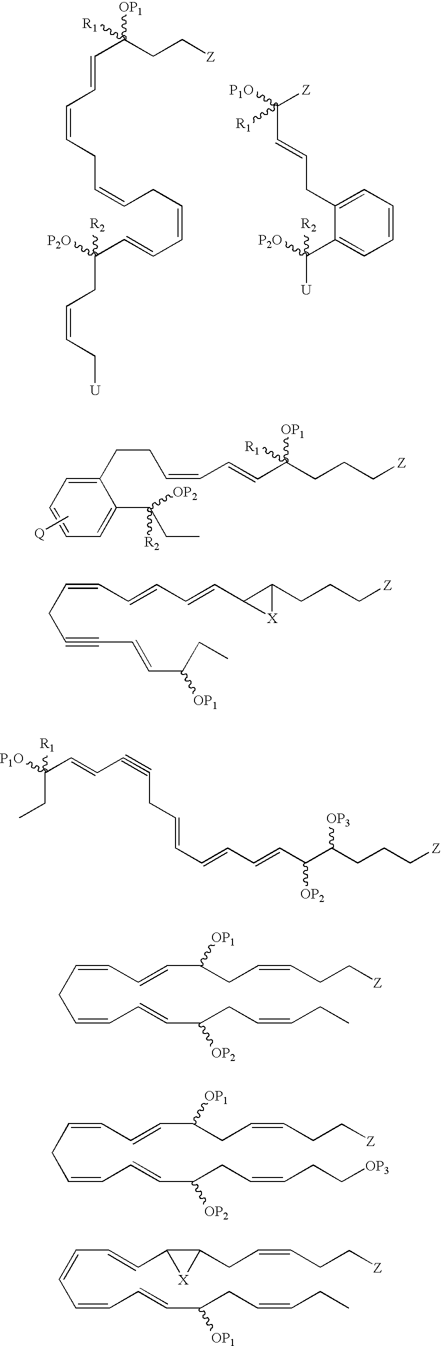 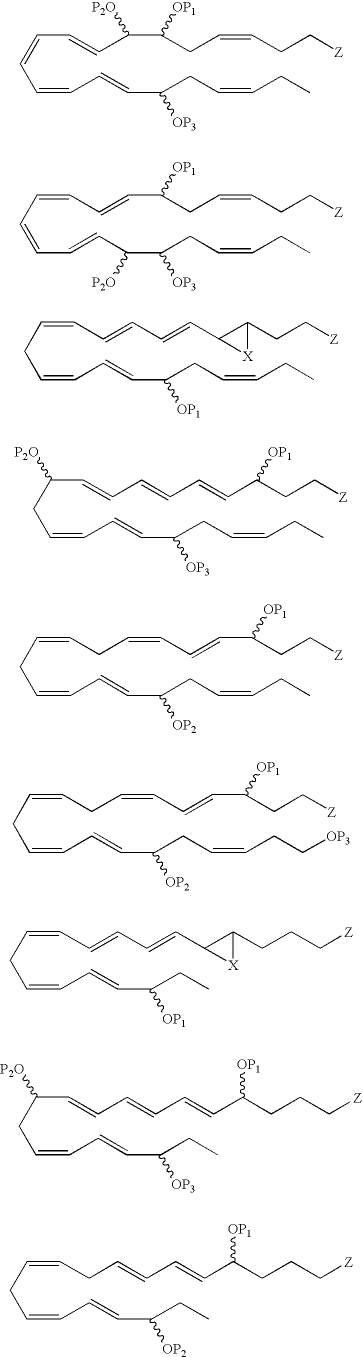 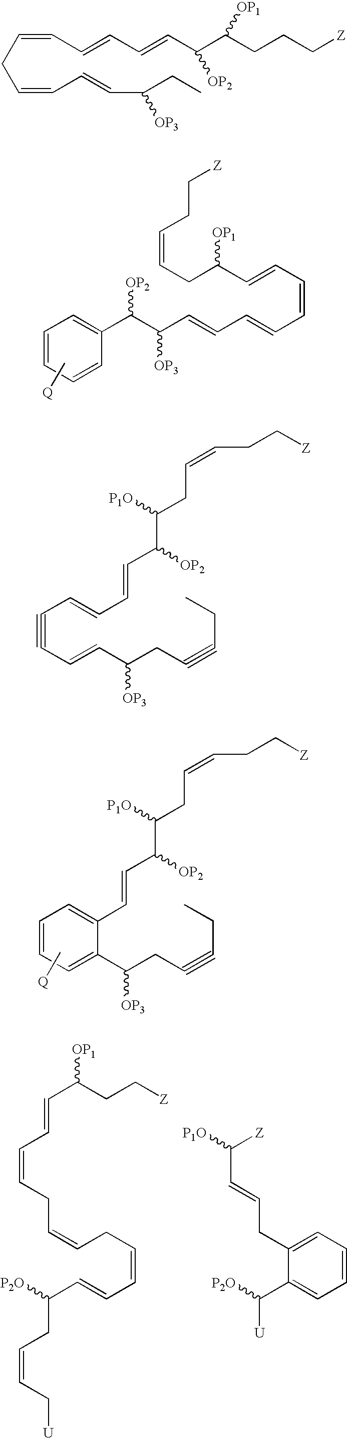 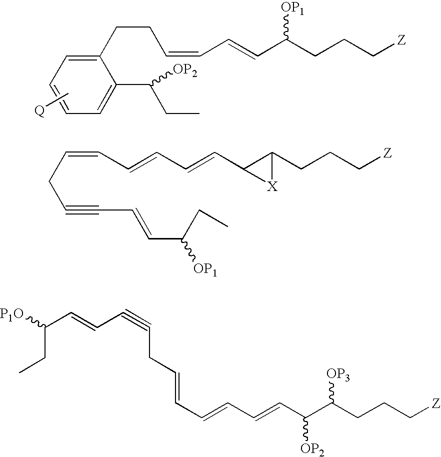
wherein P₁, P₂ and P₃, if present, each individually are protecting groups, hydrogen atoms or combinations thereof;
wherein R₁, R₂ and R₃, if present, each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof;
wherein Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;
each R^{a}, if present, is independently selected from the group consisting of hydrogen, (C1-C6)alkyl, (C3-C8)cycloalkyl, cyclohexyl, (C4-C11)cycloalkylalkyl, (C5-C10)aryl, phenyl, (C6-C16)arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;
each R^{b}, if present, is a suitable group independently selected from the group consisting of ═O, —OR^{d}, (C1-C3)haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, C(NH)NR^{c}R^{c}, C(NR^{a})NR^{c}R^{c}, —C(NOH)R^{a}, —C(NOH)NR^{c}R^{c}, —OC(O)R^{d}, —OC(O)OR, —OC(O)NR^{c}R^{c}, —OC(NH)NR^{c}R^{c}, —OC(R^{a})NR^{c}R^{c}, —NHC(O)]ₙR^{d}, —[NR^{a}C(O)]ₙR^{d}, —[NHC(O)]ₙOR^{d}, —[NR^{a}C(O)]OR d, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;
each n, independently, if present, is an integer from 0 to 3;
each R^{d}, independently, if present, is a protecting group or R^{a};
wherein X, if present, is a substituted or unsubstituted methylene, an oxygen atom, a substituted or unsubstituted nitrogen atom, or a sulfur atom;
wherein Q, if present, represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group;
wherein U, if present, is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;
wherein the double bond configurations of the compounds can be either cis or trans;
pharmaceutically acceptable salts thereof; And
a pharmaceutically acceptable carrier.

9. The method of claim 8, wherein Z is a carboxylic acid, ester, a pharmaceutically acceptable carboxylic acid salt, or prodrug thereof.

10. The method of claim 8, wherein the C-5, C-6, C-12, C-13, C-14, C-15, C-16, C-17, C-18, C-19 or C-20, each individually, if present, has an R configuration if it is a chiral center.

11. The method of claim 8, wherein the C-5, C-6, C-12, C-13, C-14, C-15, C-16, C-17, C-18, C-19 or C-20, each individually, if present, has an S configuration if it is a chiral center.

12. The method of claim 8, wherein the C-5, C-6, C-12, C-13, C-14, C-15, C-16, C-17, C-18, C-19 or C-20, each individually, if present, has an R/S configuration if it is a chiral center.

13. The method of claim 8, wherein the gastrointestinal condition is ulcerative colitis, Crohn's disease, infectious enteritis, antibiotic associative diarrhea, clostridium difficile colitis, microscopic or lymphocytic colitis, collagenous colitis, colon polyps, familial polyps, familial polyposis syndrome, Gardner's Syndrome, helicobacter pylori, irritable bowel syndrome, nonspecific diarrheal illnesses, and intestinal cancers.

14. The method of claim 8, wherein the gastrointestinal condition is gastrointestinal inflammation.

15. A packaged pharmaceutical to treat or prevent a gastrointestinal condition, comprising a compound selected from: 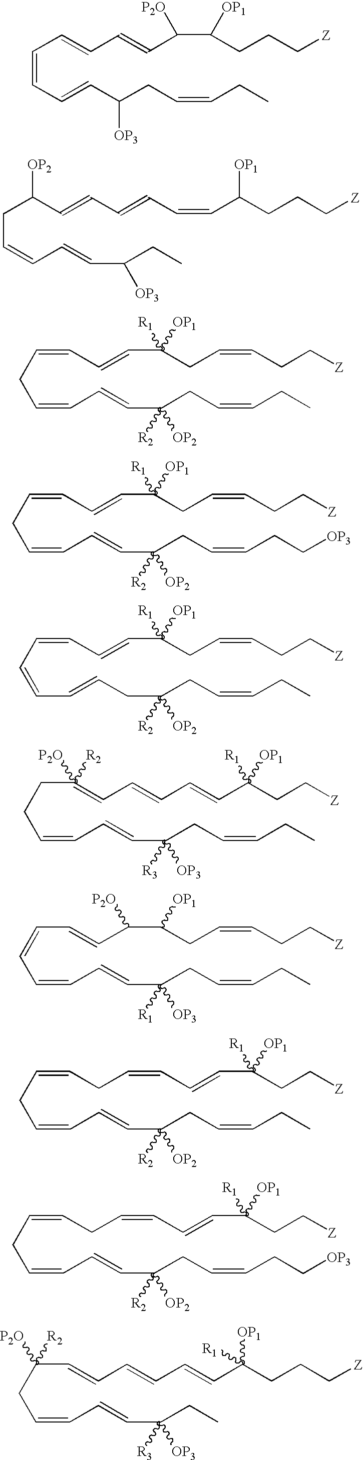 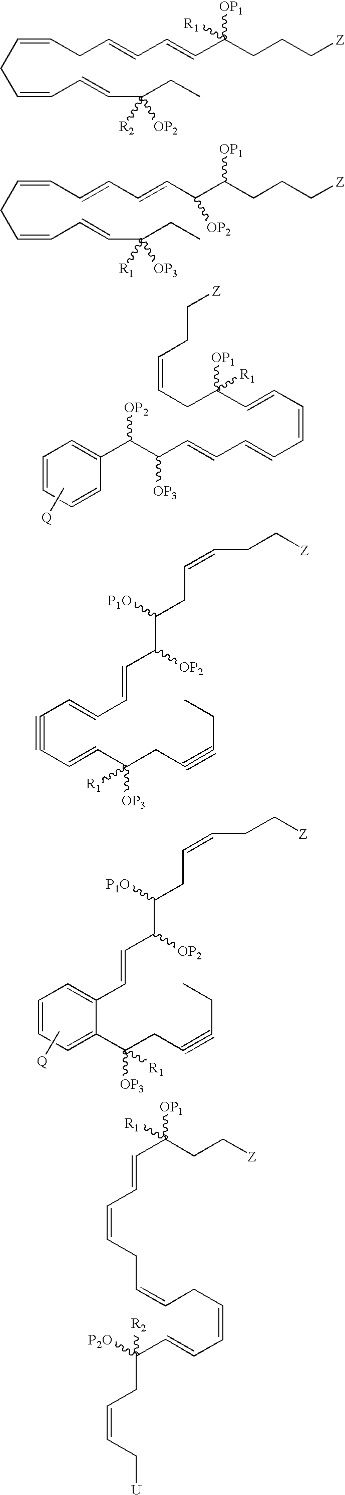 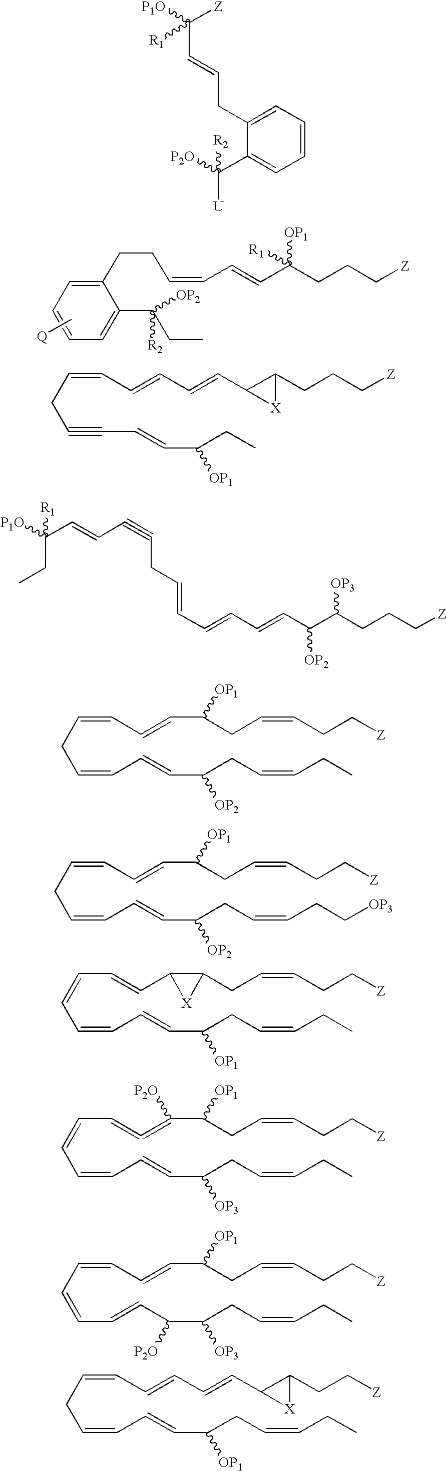 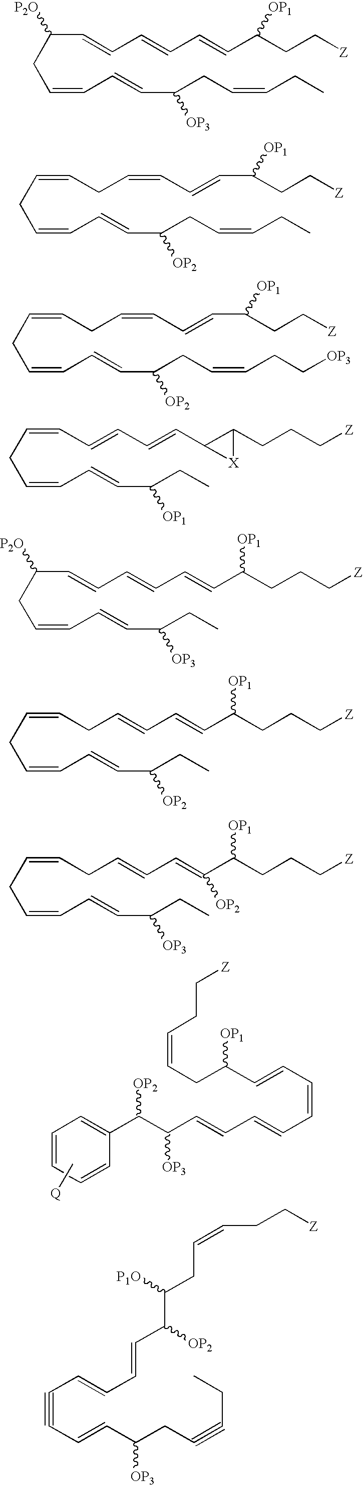 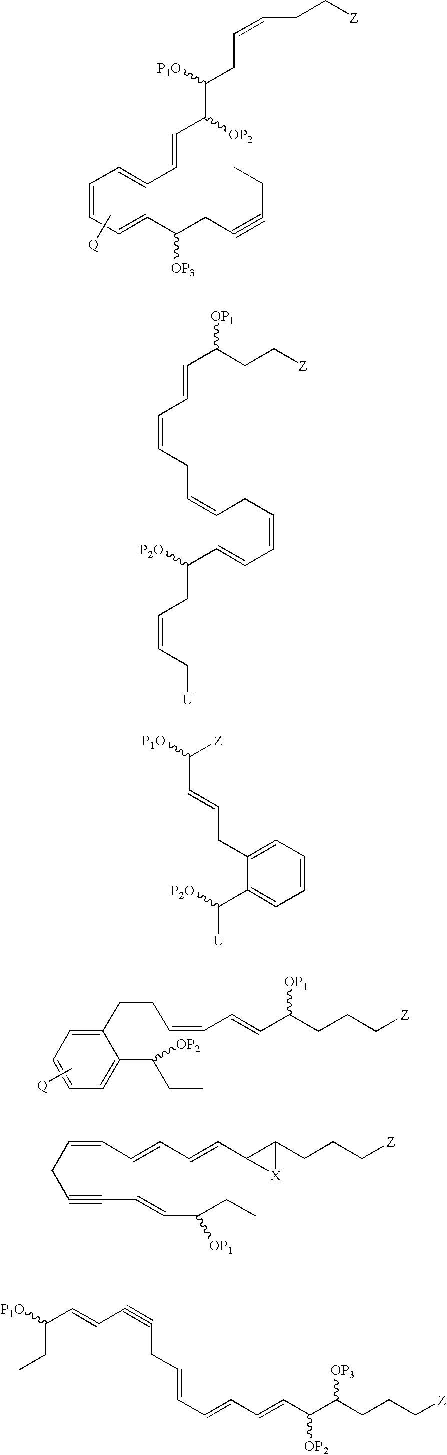
wherein P₁, P₂ and P₃, if present, each individually are protecting groups, hydrogen atoms or combinations thereof;
wherein R₁, R₂ and R₃, if present, each individually are substituted or unsubstituted, branched or unbranched alkyl groups, substituted or unsubstituted aryl groups, substituted or unsubstituted, branched or unbranched alkylaryl groups, halogen atoms, hydrogen atoms or combinations thereof;
wherein Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN;
each R^{a}, if present, is independently selected from the group consisting of hydrogen, (C1-C6)alkyl, (C3-C8)cycloalkyl, cyclohexyl, (C4-C11)cycloalkylalkyl, (C5-C10)aryl, phenyl, (C6-C16)arylalkyl, benzyl, 2-6 membered heteroalkyl, 3-8 membered cycloheteroalkyl, morpholinyl, piperazinyl, homopiperazinyl, piperidinyl, 4-11 membered cycloheteroalkylalkyl, 5-10 membered heteroaryl and 6-16 membered heteroarylalkyl;
each R^{b}, if present, is a suitable group independently selected from the group consisting of ═O, —OR^{d}, (C1-C3)haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, —[NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, if present, is independently a protecting group or R^{a}, or, alternatively, each R^{c} is taken together with the nitrogen atom to which it is bonded to form a 5 to 8-membered cycloheteroalkyl or heteroaryl which may optionally include one or more of the same or different additional heteroatoms and which may optionally be substituted with one or more of the same or different R^{a} or suitable R^{b} groups;
each n, independently, if present, is an integer from 0 to 3;
each R^{d}, independently, if present, is a protecting group or R^{a};
wherein X, if present, is a substituted or unsubstituted methylene, an oxygen atom, a substituted or unsubstituted nitrogen atom, or a sulfur atom;
wherein Q, if present, represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group;
wherein U, if present, is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;
wherein the double bond configurations of the compounds can be either cis or trans;
pharmaceutically acceptable salts thereof; and
instructions for administration of the compound, thereby treating or preventing the gastrointestinal condition.

16. The packaged pharmaceutical of claim 15, wherein Z is a carboxylic acid, ester, a pharmaceutically acceptable carboxylic acid salt, or prodrug thereof.

17. The packaged pharmaceutical of claim 15, wherein the C-5, C-6, C-12, C-13, C-14, C-15, C-16, C-17, C-18, C-19 or C-20, each individually, if present, has an R configuration if it is a chiral center.

18. The packaged pharmaceutical of claim 15, wherein the C-5, C-6, C-12, C-13, C-14, C-15, C-16, C-17, C-18, C-i9 or C-20, each individually, if present, has an S configuration if it is a chiral center.

19. The packaged pharmaceutical of claim 15, wherein the C-5, C-6, C-12, C-13, C-14, C-15, C-16, C-17, C-18, C-19 or C-20, each individually, if present, has an R/S configuration if it is a chiral center.

20. The packaged pharmaceutical of claim 15, wherein the gastrointestinal condition is ulcerative colitis, Crohn's disease, infectious enteritis, antibiotic associative diarrhea, clostridium difficile colitis, microscopic or lymphocytic colitis, collagenous colitis, colon polyps, familial polyps, familial polyposis syndrome, Gardner's Syndrome, helicobacter pylori, irritable bowel syndrome, nonspecific diarrheal illnesses, and intestinal cancers.

21. The packaged pharmaceutical of claim 15, wherein the gastrointestinal condition is gastrointestinal inflammation.
